SEQUENCE LISTING (14 sequences)

SEQ ID NO: 1 - protein, 7096 aa, Artificial Sequence [sequence omitted]

SEQ ID NO: 2 - protein, 1273 aa, Artificial Sequence
  Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 222 aa, Artificial Sequence
  Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 4 - protein, 75 aa, Artificial Sequence
  Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 5 - protein, 419 aa, Artificial Sequence
  Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 6 - protein, 7096 aa, Artificial Sequence
  Severe acute respiratory syndrome coronavirus 2
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGGAYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGGAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGGKIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQSKMSDVKCTSVVLLSVLQQLRVESSSKLWAQCVQLHNDILLAKDTTEAFEKMVSLLSVLLSMQGAVDINKLCEEMLDNRATLQAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQNNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQAGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQAVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNLAWRKAVFISPYNSQNAVASKILGLPTQTVDSSQGSECDYVIFTQTTETAHSCNVNRFNVAITRAKVGILCIMSDRDLYDKLQFTSLEIPRRNVATLQAENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQSLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQSSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

SEQ ID NO: 7 - protein, 275 aa, Artificial Sequence
  Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 8 - protein, 75 aa, Artificial Sequence; identical to SEQ ID NO: 4
  Severe acute respiratory syndrome coronavirus 2

SEQ ID NO: 9 - protein, 222 aa, Artificial Sequence; identical to SEQ ID NO: 3
  Severe acute respiratory syndrome coronavirus 2

SEQ ID NO: 10 - protein, 61 aa, Artificial Sequence
  Severe acute respiratory syndrome coronavirus 2
MFHLVDFQVTIAEILLIIMRTFKVSIWNLDYIINLIIKNLSKSLTENKYSQLDEEQPMEID

SEQ ID NO: 11 - protein, 121 aa, Artificial Sequence
  Severe acute respiratory syndrome coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFTTLCFTLKRKTE

SEQ ID NO: 12 - protein, 121 aa, Artificial Sequence
  Severe acute respiratory syndrome coronavirus 2
MKFLVFLGIITTVAAFHQECSLQSCTQHQPYVVDDPCPIHFYSKWYIRVGARKSAPLIELCVDEAGSKSPIQYIDIGNYTVSCSPFTINCQEPKLGSLVVRCSFYEDFLEYHDVRVVLDFI

SEQ ID NO: 13 - protein, 419 aa, Artificial Sequence; identical to SEQ ID NO: 5
  Severe acute respiratory syndrome coronavirus 2

SEQ ID NO: 14 - protein, 38 aa, Artificial Sequence
  Severe acute respiratory syndrome coronavirus 2
MGYINVFAFPFTIYSLLLCRMNSRNYIAQVDVVNFNLT

(19) United States
(12) Patent Application Publication
(10) Pub. No.: US 2022/0254458 A1
(43) Pub. Date: Aug. 11, 2022

(54) LINKAGE OF A POINT OF CARE (POC) TESTING MEDIA AND A TEST RESULT FORM USING IMAGE ANALYSIS

(71) Applicant: BioReference Health, LLC, Elmwood Park, NJ (US)

(72) Inventors: John MOONEY, Berkeley Heights, NJ (US); Teresita PINON, Edgewater, NJ (US); Mashal ZIA, Randolph, NJ (US)

(73) Assignee: BioReference Health, LLC, Elmwood Park, NJ (US)

(21) Appl. No.: 17/590,517

(22) Filed: Feb. 1, 2022

Related U.S. Application Data
(60) Provisional application No. 63/146,502, filed on Feb. 5, 2021.

(51) Int. Cl. G16H 10/40; G16H 10/60; G16H 50/20; G06V 30/412

(52) U.S. Cl. CPC G16H 10/40; G06V 30/412; G16H 50/20; G16H 10/60

(57) ABSTRACT

Disclosed herein are system, method, and computer program product embodiments for linking a point of care (POC) testing media and a test result form using image analysis. In some embodiments, a server receives an image of a diagnostic test result form and a POC diagnostic testing media. The server confirms that the diagnostic test result form and the POC diagnostic testing media are positioned in a predetermined position such that the patient's information and the patient identifier are visible in the image. Furthermore, the server verifies that the diagnostic test result form and the POC diagnostic testing media correspond to the patient. The server extracts the form ID and one or more values on the diagnostic test result form from the image. The server transmits the patient information, the patient identifier, the form ID, and the one or more values to a client device.

### CROSS-REFERENCE TO RELATED APPLICATIONS

This application claims priority to U.S. Provisional Application No. 63/146,502, filed on Feb. 5, 2021, the contents of which are incorporated herein by reference in their entirety.

### REFERENCE TO SEQUENCE LISTING SUBMITTED ELECTRONICALLY

The content of the electronically submitted sequence listing (Name: 2611_064PC02_Seqlisting.txt; Size: 156,606 Bytes; and Date of Creation: Jan. 28, 2022) is herein incorporated by reference in its entirety.

### BACKGROUND

SARS-CoV-2 is a positive-sense single-stranded ribonucleic acid (RNA) virus with genetic similarity to bat coronaviruses. SARS-CoV-2 was first isolated in January 2020 from patients in Wuhan, China (Hui et al., (2020)). The SARS-CoV-2 viral pathogen (also referred to as the COVID-19 pathogen) has caused a global pandemic with millions of people infected throughout the world. In the United States alone, as of February 2021, over 430,000 people have died of the disease, with over 24 million infected. Globally, many more people have been infected and have died from the COVID-19 pathogen. The virus has caused significant disruptions in the global economy and in human suffering and has altered the course of daily behavior on a global basis.

There have been rapid advances in vaccines, therapeutics, and treatment methods throughout the pandemic. The testing to date ranges from lab-based service providers in a centralized laboratory setting to point of care (POC) devices that detect the virus. Lab-based service providers have been critical in the testing of millions of individuals for potential infection with COVID-19. While these services are essential and provide results to patients and health care providers in a relatively rapid manner, there is a continual need to obtain faster and more reliable data from the POC settings or home or business settings. Thus, there is an unmet medical need to secure diagnostic results for COVID-19 and other infectious diseases from any location using a POC device. However, POC testing systems and methods suffer from various technological problems that have not been adequately addressed. The first technological problem is orders integration, the second technological problem is barcode reading capability, and the last technological problem is results integration.

First, POC systems and methods do not automatically provide information about the laboratory order or request. Thus, POC systems do not capture, store or send important information about the ordering provider, patient, or specimen.

Second, POC systems and methods do not offer an automatic reading of specimen barcodes. As a result, there are no automatic safeguards in place to prevent patient misidentification or specimen switching problems.

Finally, POC systems and methods do not have the ability to automatically transmit the test results to a laboratory information system (LIS) or other central lab storage and processing facility. As a result, the test results must be manually entered into the LIS. This can increase the labor required, the turnaround time, and error rates.

### SUMMARY

Embodiments herein relate to POC diagnostic systems and methods of using such systems to generate patient-specific results. An embodiment herein includes a system of a plurality of different POC diagnostic testing devices and diagnostic test assays connected by a computing device that captures patient-specific requisitions and results and information contained therein and further translates the images captured by optical character recognition to provide results and/or interpreted results. An embodiment is particularly useful to capture data generated from many different diagnostic devices. The diagnostic devices conduct tests for stakeholders such as sports teams, businesses, or any defined group that, for example, includes cruise line passengers and their managers. A mobile application on a smartphone can generate the results and transmit the results to a central laboratory for results production, communication, and/or management.

Some embodiments herein include a computer-implemented method for linking a POC testing media and a diagnostic test result form. The computer-implemented method includes receiving an image of the diagnostic test result form and a POC diagnostic testing media. The diagnostic test result form includes a POC diagnostic test result, form ID, and a patient identifier. The POC diagnostic test result is generated based on a POC diagnostic test being performed on a patient using a POC diagnostic testing device. The POC diagnostic testing media includes the patient's information and specimen information. The computer-implemented method further includes confirming that the diagnostic test result form and the POC diagnostic testing media are positioned in a predetermined position such that the patient's information and the patient identifier are visible in the image. Furthermore, the computer-implemented method includes verifying that the diagnostic test result form and the POC diagnostic testing media correspond to the patient, and extracting the form ID and values on the diagnostic test result form from the image. The values correspond with attributes of the POC diagnostic test result. Moreover, the computer-implemented method includes transmitting the patient information, the patient identifier, the form ID, and the values to a client device.

Some embodiments herein include a system for linking a POC testing media and a diagnostic test result form. The system includes a memory and at least one processor coupled to the memory. The at least processor is configured to receive an image of the diagnostic test result form and a POC diagnostic testing media. The diagnostic test result form includes a POC diagnostic test result, form ID, and a patient identifier. The POC diagnostic test result is generated based on a POC diagnostic test being performed on a patient using a POC diagnostic testing device. The POC diagnostic testing media includes the patient's information and specimen information. The processor is further configured to confirm that the diagnostic test result form and the POC diagnostic testing media are positioned in a predetermined position such that the patient's information and the patient identifier are visible in the image. Furthermore, processor is further configured to verify that the diagnostic test result form and the POC diagnostic testing media correspond to the patient, and extracting the form ID and values on the diagnostic test result form from the image. The values correspond with attributes of the POC diagnostic test result. Moreover, the processor is further configured to transmit the patient information, the patient identifier, the form ID, and the values to a client device.

Some embodiments herein include a non-transitory computer-readable medium. The non-transitory computer readable media includes instructions stored thereon. Execution of the instructions, by one or more processors of a device cause the one or more processors to perform operations. The operations include receiving an image of the diagnostic test result form and a POC diagnostic testing media. The diagnostic test result form includes a POC diagnostic test result, form ID, and a patient identifier. The POC diagnostic test result is generated based on a POC diagnostic test being performed on a patient using a POC diagnostic testing device. The POC diagnostic testing media includes the patient's information and specimen information. The operations further include confirming that the diagnostic test result form and the POC diagnostic testing media are positioned in a predetermined position such that the patient's information and the patient identifier are visible in the image. Furthermore, the operations include verifying that the diagnostic test result form and the POC diagnostic testing media correspond to the patient, and extracting the form ID and values on the diagnostic test result form from the image. The values correspond with attributes of the POC diagnostic test result. Moreover, the he operations include transmitting the patient information, the patient identifier, the form ID, and the values to a client device.

Some embodiments herein include a device configured to interpret a POC diagnostic test result. The device includes a memory, a camera, and a processor coupled to the memory and the camera. The processor is configured to cause the camera to capture an image of a diagnostic test result form and a POC diagnostic testing media. The diagnostic test result form includes a POC diagnostic test result, form ID, and a patient identifier. The POC diagnostic test result is generated based on a POC diagnostic test being performed on a patient using a POC diagnostic testing device. The POC diagnostic testing media includes the patient's information and specimen information. The processor transmits the image to a web-service to extract data from the diagnostic test result form and the POC diagnostic testing media and receive the data from the web-service. The data includes the patient information, the patient identifier, the form ID, and one or more values corresponding with one or more attributes of the POC diagnostic test result. The processor stores the data in a local storage device. Furthermore, the processor receives an identification number, and date of birth information corresponding to the patient. The processor retrieves an order for the POC diagnostic test conducted on the patient using the identification number and the date of birth information, and determines a type of POC diagnostic test based on the form ID. Moreover, the processor determines a relationship between the one or more attributes based on the type of POC diagnostic test. The processor interprets the POC diagnostic test result based on the one or more values, and the relationship between the one or more attributes, and outputs the interpreted POC diagnostic test result.

In each of the embodiments above, there may be more particular features of each element which are further disclosed and described herein.

### BRIEF DESCRIPTION OF THE DRAWINGS

FIG. 1 is a block diagram of a system for linking a diagnostic test result form to a POC diagnostic testing media, according to some embodiments.

FIG. 2A is a flowchart illustrating a process for identifying and extracting information on a diagnostic test result form and POC diagnostic testing media, according to some embodiments.

FIG. 2B is a flowchart illustrating a process for outputting the POC diagnostic test result, according to some embodiments.

FIG. 3A illustrates a diagram of the various technologies used in the POC platforms currently authorized by the FDA under emergency use authorization, according to some embodiments.

FIGS. 3B-3C illustrate an example POC platform, according to some embodiments.

FIGS. 4A, 4B and 4C illustrate graphical user interfaces (GUI) of an application for capturing POC diagnostic test results, according to some embodiments.

FIGS. 5A and 5B illustrate GUIs of the application capturing POC diagnostic test results, according to some embodiments.

FIG. 6 provides a schematic description of preparation for testing and results capture, according to some embodiments.

FIG. 7 is a flowchart illustrating a method for linking a POC testing media and a diagnostic test result form, according to some embodiments.

FIG. 8 is a flowchart illustrating a method for interpreting a POC diagnostic test result, according to some embodiments.

FIG. 9 is an example computer system useful for implementing various embodiments.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, device, method, and/or computer program product embodiments, and/or combinations and sub-combinations thereof for performing POC diagnostic testing for COVID-19. Embodiments herein further provide for linking a POC testing media and a test result form using image analysis.

SARS-CoV-2 is a positive-sense single-stranded ribonucleic acid (RNA) virus with genetic similarity to bat coronaviruses. SARS-CoV-2 was first isolated in January 2020 from patients in Wuhan, China (Hui et al., (2020) supra. SARS-CoV-2 is a species of the Betacoronavirus genus, which is part of the Orthocornoavirinae subfamily, which, in turn, is part of the Coronaviridae family. The complete genome sequences of different strains and variants of SARS-CoV-2 have been determined and are publicly available online at the GenBank database of the National Center for Biotechnology Information (NCBI) website (ncbi.nlm.nih.gov). One complete genome sequence, which is considered as the reference genome sequence, is available as GenBank Accession No. NC_045512 (version NC_045512.2 as of Jan. 17, 2020). This GenBank record, which also provides the amino acid sequence of the encoded proteins, are provided herein as SEQ ID NO: 1. The SARS-CoV-2 genome sequence encodes several proteins, including, but not limited to a spike (S) protein (SEQ ID NO: 2), membrane protein (SEQ ID NO: 3), envelope protein (SEQ ID NO: 4), or nucleocapsid protein (SEQ ID NO: 5). Other SARS CoV-2 proteins include but are not limited to ORF lab (open reading frame) polyprotein (QIQ50091.1) (SEQ ID NO: 6), ORF3a protein (QIQ50093.1) (SEQ ID NO: 7), envelope protein (QIQ50094.1) (SEQ ID NO: 8), membrane glycoprotein (QIQ50095.1) (SEQ ID NO: 9), ORF6 protein (QIQ50096.1) (SEQ ID NO: 10), ORF7a protein (QIQ50097.1) (SEQ ID NO: 11), ORF8 protein (QIQ50098.1) (SEQ ID NO: 12), nucleocapsid protein (QIQ50099.1) (SEQ ID NO: 13) and ORF10 protein (QIQ50100.1) (SEQ ID NO: 14). ORF1a encodes polyprotein pp1a, and via a programmed frameshift, the combination of ORF1a and ORF1b encodes polyprotein pp1ab (Jungreis et al., 2021). ORF1a encodes polyprotein pp1a, and via a programmed frameshift, the combination of ORF1a and ORF1b encodes polyprotein pp1ab (Jungreis et al., 2021). As the virus mutates and evolves, various other sequences for each of the proteins and RNA encoding them are also detected after such genes have been sequenced. These variants include, but are not limited to, the Alpha, Beta, Gamma, Delta, and Omicron variants, as designated by the World Health Organization (WHO). These variants were previously referred to by the country in which they were first identified (e.g., Alpha “UK,” Beta “South Africa,” and Gamma “Brazil”). The associated Phylogenetic Assignment of Named Global Outbreak (PANGO) parent lineages are as follows, B.1.1.7, B.1.351, B.1.1.28.1 (alias is P.1), B.1.617.2, B.1.1.529, respectively. See, e.g., cov-lineages.org/index.html#global_reports. As described in Rambaut et al., 2020, when the lineage hierarchy reaches a certain depth, lineage names are given an alias to prevent them from becoming infinitely long.

A non-exhaustive list of single nucleotide polymorphisms (SNPs) associated with exemplary SARS-CoV-2 variants, which at one point were designated variants of concern (VOCs; see e.g., https://www.cdc.gov/coronavirus/2019-ncov/variants/variant-classifications.html) are displayed in Table 1. See e.g., https://cov-lineages.org/index.html#global_reports.

| | | Defining, non- | |
|---|---|---|---|
| | | exhaustive, SNPs | |
| | PANGO | relative to the | |
| WHO | lineage | SARS-CoV-2 | |
| label | (parent) | reference sequence | Lineage assignment |
| Omicron | B.1.1.529 | del: 6513: 3 | In certain aspects, B.1.1.529 is assigned |
| | | del: 11283: 9 | to sequences with at least 32 of the 47 |
| | | nuc: C241T | defining B.1.1.529 SNPs. See e.g., https: //cov- |
| | | ORF1A: K856R | lineages.org/global_report_B.1.1.529.html. |
| | | nuc: C3037T | |
| | | nuc: T5386G | |
| | | ORF1a: A2710T | |
| | | ORF1a: T3255I | |
| | | ORF1a: P3395H | |
| | | ORF1a: I3758V | |
| | | nuc: T13195C | |
| | | ORF1b: P314L | |
| | | nuc: C15240T | |
| | | ORF1b: I1566V | |
| | | S: A67V | |
| | | S: T95I | |
| | | S: G339D | |
| | | S: S371L | |
| | | S: S373P | |
| | | S: K417N | |
| | | S: N440K | |
| | | S: G446S | |
| | | S: S477N | |
| | | S: T478K | |
| | | S: E484A | |
| | | S: Q493R | |
| | | S: G496S | |
| | | S: Q498R | |
| | | S: N501Y | |
| | | S: T547K | |
| | | S: D614G | |
| | | S: H655Y | |
| | | S: N679K | |
| | | S: P681H | |
| | | S: N764K | |
| | | S: D796Y | |
| | | S: N856K | |
| | | S: Q954H | |
| | | S: N969K | |
| | | nuc: C25000T | |
| | | E: T9I | |
| | | M: D3G | |
| | | M: Q19E | |
| | | M: A63T | |
| | | nuc: A27259C | |
| | | nuc: C27807T | |
| | | N: RG203KR | |
| Delta | B.1.617.2 | S: T19R | In certain aspects, B.1.617.2 is assigned |
| | | S: L452R | to sequences with at least 5 of the |
| | | S: T478K | defining B.1.617.2 SNPs. See e.g., https: //cov- |
| | | S: P681R | lineages.org/global_report_B.1.617.2.html. |
| | | S: D950N | |
| | | ORF3a: S26L | |
| | | M: I82T | |
| | | ORF7a: V82A | |
| | | ORF7a: T120I | |
| | | N: D63G | |
| | | N: R203M | |
| | | N: D377Y | |
| Gamma | B.1.1.28.1 | ORF1ab: S1188L | In certain aspects, B.1.1.28.1 (alias P.1) |
| | (alias is P.1) | ORF1ab: K1795Q | is assigned to sequences with at least |
| | | ORF1ab: E5665D | 10 of the 17 defining SNPs. See e.g., https.//cov- |
| | | del: 11288: 9 | lineages.org/global_report_P_1.html and |
| | | S: L18F | https: //virological.org/t/genomic- |
| | | S: T20N | characterisation-of-an-emergent-sars-cov-2 |
| | | S: P26S | -lineage-in-manaus-preliminary-findings/586. |
| | | S: D138Y | |
| | | S: R190S | |
| | | S: K417T | |
| | | S: E484K | |
| | | S: N501Y | |
| | | S: H655Y | |
| | | S: T1027I | |
| | | ORF3a: G174C | |
| | | ORF8: E92K | |
| | | N: P80R | |
| Beta | B.1.351 | E: P71L | In certain aspects, B.1.351 is assigned |
| | | N: T205I | to sequences with at least 5 of the 9 |
| | | ORF1a: K1655N | defining B.1.351 SNPs. See e.g., https: //cov- |
| | | S: D80A | lineages.org/global_report_B.1.351.html. |
| | | S: D215G | |
| | | S: K417N | |
| | | S: A701V | |
| | | S: N501Y | |
| | | S: E484K | |
| Alpha | B.1.1.7 | ORF1ab: T1001I | In certain aspects, B.1.1.7 is assigned |
| | | ORF1ab: A1708D | to sequences with at least 5 of the 17 |
| | | ORF1ab: I2230T | defining B.1.1.7 SNPs. See e.g., http: //cov- |
| | | del: 11288: 9 | lineages.org/global_report_B.1.1.7.html and |
| | | del: 21765: 6 | https: //virological.org/t/preliminary- |
| | | del: 21991: 3 | genomic-characterisation-of-an-emergent- |
| | | S: N501Y | sars-cov-2-lineage-in-the-uk-defined-by- |
| | | S: A570D | a-novel-set-of-spike-mutations/563. |
| | | S: P681H | |
| | | S: T716I | |
| | | S: S982A | |
| | | S: D1118H | |
| | | ORF8: Q27* | |
| | | ORF8: R52I | |
| | | ORF8: Y73C | |
| | | N: D3L | |
| | | N: S235F | |

The defining, non-exhaustive, SNPs refer to amino acid positions unless specified by “nuc,” which refers to the nucleotide position. The deletions (“del”) refer to the nucleotide position. The following abbreviations are also used in Table 1 to refer to protein domains: membrane (M) protein, envelope (E) protein, and nucleocapsid (N) protein.

The SARS-CoV-2 viral pathogen (also referred to as the COVID-19 pathogen) has caused a global pandemic with millions of people infected throughout the world. In the United States alone, as of February 2021, over 430,000 people have died of the disease, with over 24 million infected. Globally, many more people have been infected and have died from the COVID-19 pathogen. The virus has caused significant disruptions in the global economy and in human suffering and has altered the course of daily behavior on a global basis.

There have been rapid advances in vaccines and in therapeutics and treatment methods throughout the pandemic. In parallel with such advances, diagnostic methods have also advanced over the course of the pandemic. Specialized and focused methods to test groups of individuals to permit rapid diagnosis and treatment for vulnerable populations such as essential workers or especially vulnerable individuals and groups such as black and brown or American Indian populations are clearly needed. In addition, economic and societal considerations that permit sports teams to play at the professional, college, and other levels has also created the need for rapid testing, diagnosis, and contact tracing as central objectives to ensure the safety of infected and non-infected individuals or groups of individuals. In addition, the critical importance of education at all levels from early childhood to college has necessitated the need for high throughput testing and analytics that can, in real-time, provide information for treatment decisions as well as economic and business decisions.

Diagnostic methods to detect SARS-CoV-2 developed rapidly following the initial discovery of the disease in Wuhan, China. These methods generally range from reverse transcription (RT) polymerase chain reaction (PCR) amplification of the viral genome to antibody detection methods using a myriad of viral components or antibodies thereto to test for the pathogen. The testing to date ranges from lab-based service providers in a centralized laboratory setting to POC devices that detect the virus. Lab-based service providers have been critical in the testing of millions of individuals that are potentially infected with COVID-19. While these services are essential and important and provide results to the patients and health care providers in a relatively rapid manner, there is a continual need to obtain faster and reliable data from POC settings or home or business settings. Thus, there is an unmet medical need to secure diagnostic results for COVID-19 and other infectious diseases from any location using a POC device and to automate the results from such devices in real-time to then provide such information as quickly as possible to the potentially infected individual and to the healthcare provider and other persons or groups having a need to know the results. However, point-of-care testing systems and methods suffer from various technological problems that have not been adequately addressed. The first technological problem is orders integration, the second technological problem is barcode reading capability, and the last technological problem is results integration.

First, POC systems and methods do not automatically provide information about the laboratory order or request. Thus, POC systems and methods do not capture, store or send important information about the ordering provider, patient, or specimen. By contrast, larger medical laboratory testing instruments often have or offer the ability to automatically receive/retrieve order information from, for example, a laboratory information system via a software interface such as a data stream (e.g., ASTM, HL7) or application programming interface (API). Data contained in these interfaces typically has information about the patient, the ordering provider, the test to be performed, and some important dates (e.g., date specimen was collected) or other necessary information.

Second, POC systems and methods do not offer an automatic reading of specimen barcodes. As a result, there are no automatic safeguards in place to prevent patient misidentification or switching problems. By contrast, larger laboratory instruments outside the POC setting typically have the ability or capacity to automatically read specimen barcodes and store the resulting test results.

Finally, POC systems and methods do not have the ability to automatically transmit the test results to a laboratory information system (LIS) or other central lab storage and processing facility. As a result, the test results must be manually entered into the LIS. This can increase the labor required, the turnaround time, and error rates. By contrast, larger laboratory testing instruments generally have the ability to automatically transmit the results to the LIS through software interfaces such as data streams or APIs. Data contained in these interfaces typically contains information about the patient, the ordering provider, test results, the performing instrument, and key dates.

Embodiments herein solve these technical problems by capturing data and/or results from connected or disconnected POC diagnostic testing devices (e.g., home test kits for COVID-19 or other pathogen detection results). In some embodiments, a server receives an image of a diagnostic test result form and a POC diagnostic testing media. The diagnostic test result form comprises a POC diagnostic test result generated based on a POC diagnostic test being performed on a patient using a POC diagnostic testing device and a patient identifier. The POC diagnostic testing media comprises the patient's information and specimen information. The server confirms that the diagnostic test result form and the POC diagnostic testing media are positioned in a predetermined position such that the patient's information and the patient identifier are visible in the image. Furthermore, the server verifies that the diagnostic test result form and the POC diagnostic testing media correspond to the patient. The server extracts the form ID and one or more values on the diagnostic test result form from the image. The one or more values correspond with one or more attributes tested during the POC diagnostic test. The server transmits the patient information, the patient identifier, the form ID, and the one or more values to a client device.

Embodiments herein provide for linking the POC diagnostic testing media and a diagnostic test result form. This allows for uploading and verifying POC diagnostic test data to a centralized location by capturing an image of the POC diagnostic test result form and the POC diagnostic testing media. To this end, the embodiments herein eliminate the need to manually input the POC diagnostic test result. Furthermore, the embodiments herein avoid errors in inputting the POC diagnostic test result data. Additionally, the embodiments herein avoid inaccurately linking the POC diagnostic test result form to the POC diagnostic testing media.

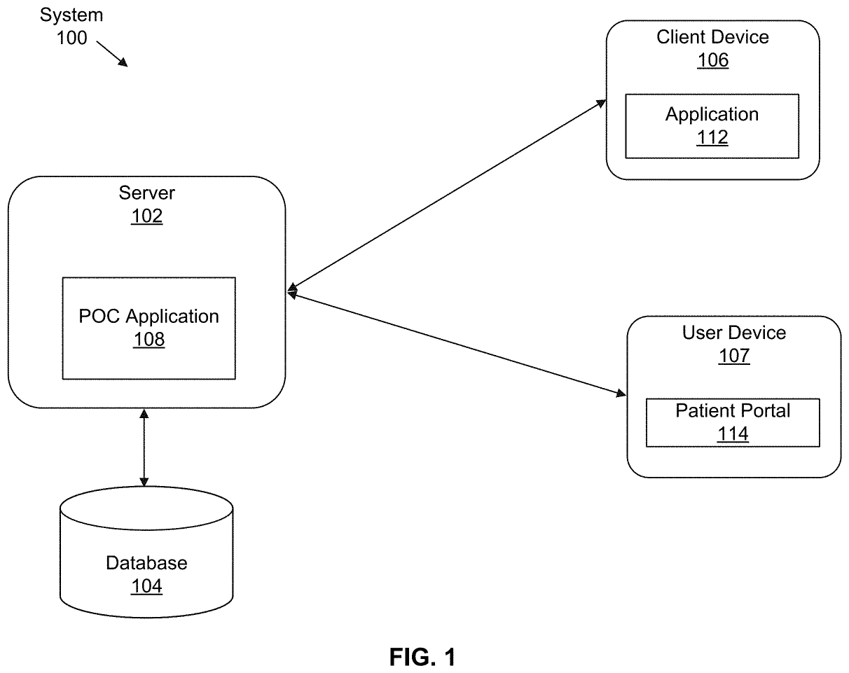

FIG. 1 is a block diagram of a system for linking a diagnostic test result form to a POC diagnostic testing media, according to some embodiments. In some embodiments, system 100 may include server 102, database 104, client device 106, and user device 107. The devices in the architecture can be connected through wired connections, wireless connections, or a combination of wired and wireless connections.

As an example, the devices can be connected through a network. The network can be an ad hoc network, an intranet, an extranet, a virtual private network (VPN), a local area network (LAN), a wireless LAN (WLAN), a wide area network (WAN), a wireless wide area network (WWAN), a metropolitan area network (MAN), a portion of the Internet, a portion of the Public Switched Telephone Network (PSTN), a cellular telephone network, a wireless network, a WiFi network, a WiMax network, any other type of network, or a combination of two or more such networks.

Server 102 may reside wholly or partially in a cloud computing environment. Alternatively, server 102 may reside outside a cloud computing environment. Furthermore, server 102 may include a POC application 108. POC application 108 may be configured to link a diagnostic test result form to a POC diagnostic testing media, validate the POC diagnostic test result on the diagnostic test result form, and store/retrieve the POC diagnostic test result using an image of the diagnostic test result form and POC diagnostic testing media received from client device 106.

Database 104 may be one or more data storage devices. Database 104 may be configured to store structured and non-structured data. Furthermore, database 104 may be configured to store POC diagnostic test results and images of the POC diagnostic test result form and POC diagnostic testing media. Database 104 may be associated with a specific entity, such as a laboratory, medical facility, medical provider, insurance company, etc. Therefore, database 104 may store data specifically associated with the entity. The data may include POC diagnostic test results and images of the POC diagnostic test result for POC diagnostic tests performed by the entity. Alternatively, database 104 may store data associated with multiple entities.

Client device 106 may include a camera 110 and application 112. Camera 110 may be configured to capture images. Furthermore, application 112 may be used to transmit images of the diagnostic test result form and POC diagnostic testing media. For example, a user may interact with client device 106 to launch application 112. Application 112 may cause camera 110 to become operational based on user input. Camera 110 may continuously capture image frames within the field of view of camera 110. The diagnostic test result form and POC diagnostic testing media may be within the field of view of camera 110. As such, camera 110 may capture an image of the diagnostic test result form and POC diagnostic testing media in response to user input or automatically. Application 112 may transmit the captured image to server 102.

In some embodiments, a POC diagnostic test may be performed on a patient using a POC diagnostic testing device. In some embodiments, the POC diagnostic testing device can be a connected or disconnected home test kit. The results from these POC diagnostic testing devices (e.g., home test kits) can be added to the information flow from lab-based test results. For example, POC diagnostic testing devices, such as ELLUME can capture positive, negative, or invalid results. For example, these positive, negative, or invalid results from a home testing kit can be captured using an image capture device and processed by application 112 executing on client device 106.

The results for a single patient or a stakeholder group (e.g., for a team, cruise passenger list, or a particular business group) can be collectively captured. Embodiments herein relate to capturing this collective data from disconnected POC diagnostic testing devices (e.g., home test kits) to provide both individual and collective results to stakeholders.

POC diagnostic testing devices that do not connect to any communications network, such as the Internet (often referred to as offline POC diagnostic testing devices), can suffer from the above technological problems. This is because these POC diagnostic testing devices are unable to consistently and accurately link test results to particular patients and the POC diagnostic testing media associated with those particular patients. Moreover, these POC diagnostic testing devices are unable to package their test results in a form that allows for the test result validation and confirmation of patient identity. The above-mentioned technological problems can be technologically solved by integrating an offline POC diagnostic testing device with a laboratory information system using an electronic device.

A POC diagnostic testing device that is, for the most part, a disconnected analog device that, when combined with client device 106, becomes capable of performing additional operations. For example, client device 106 can be configured to be capable of receiving, interpreting, and transmitting data, results, and information by and through operation of an image capture means/Artificial Intelligence (AI) engine as described and disclosed by embodiments herein. A POC diagnostic testing device can include any portable testing device or card that provides a test result for a target pathogen or an antibody to a pathogen. A POC diagnostic testing device can range from a simple paper testing kits to a sophisticated microfluidic POC system.

### Examples of POC Testing Devices

The information processed from POC diagnostic testing devices can further include genetic information of the virus or particular infection and mutations thereof as well as de-identified patient data. There is an unmet need to provide and facilitate a high quality, high throughput process to collect diagnostic information such as but not limited to, COVID-19 results from the disconnected POC testing platforms and to integrate and process such disconnected information while being Clinical Laboratory Improvement Amendments (CLIA) and/or Health Insurance Portability and Accountability Act (HIPAA) compliant. In addition, the process of obtaining such disconnected information from POC diagnostic testing devices currently in use, or coming on line, and inputting it into a centralized data center, permits quality review while improving the customer experience and with seamless integration of various systems, including, but not limited to, customer/clinical systems, verification and security systems, and consumer platforms and health agencies. As would be appreciated by a person of ordinary skill in the art, the POC devices can include cartridges and tests that test for other viral or bacterial pathogens.

Clinical assays or diagnostic methods to detect SARS-Cov-2 may employ a number of known tests to generate the results from the POC devices to input into digital transportable media. The assays can include tests based upon real-time PCR reverse transcription PCR (rRT-PCR) that uses probes and primers of the genes in SARs-Cov-2 inclusive of the E, N, nsp12 (RNA dependent RNA polymerase; RdRp genes)-the SARS-CoV-2-RdRp-P2 assay (Corman, V. M. et al. (2020) “Detection of 2019 Novel coronavirus (2019-nCoV) By Real-Time RT-PCR,” Eurosurveill. 25(3):200045 and subsequent more specific and sensitive tests developed throughout 2020 and 2021. These tests may utilize probes targeting the SARS-CoV-2 N gene (Won, J et al. (2020) “Development of a Laboratory-Safe And Low-Cost Detection Protocol for SARS-CoV-2 of the Coronavirus Disease 2019 (COVID-19),” Exp. Neurobiol. 29(2) doi: 10.5607/en20009); the E gene (Pfefferle, S. et al (2020) (“Evaluation of a quantitative RT-PCR Assay for the detection of the emerging coronavirus SARS-CoV-2 using a high throughput system,” Eurosurveill. 25(9) doi: 10.2807/1560-7917.ES.2020.25.9.2000152); the structural S and N genes and non-structural genes such as the RdRp gene and ORF lab real-time reverse transcriptase PCR assays which target the RNA dependent RNA polymerase (RdRp)/helicase (Hel), spike (S) and nucleocapsid (N) genes as reported by Chan, J. F. et al (2020) (“Improved Molecular Diagnosis of COVID-19 by the novel, highly sensitive and specific COVID-19-rDrp/Hel Real-Time Reverse Transcription Polymerase Chain Reaction Assay Validated in Vitro and with Clinical Specimens,” J. Clin. Microbiol. JCM.00310-20.doi: 10.1128/JCM.00310-20). Clustered Regularly Interspaced Short Palindromic Repeats (CRISPR)-based assays (see e.g., Huang Z, et al. Ultra-sensitive and high-throughput CRISPR-powered COVID-19 diagnosis. Biosens. Bioelectron. 2020;164:112316. doi: 10.1016/j.bios.2020.112316) and loop-mediated isothermal amplification (LAMP)-based diagnostics (see e.g., Kitagawa Y, et al. Evaluation of rapid diagnosis of novel coronavirus disease (COVID-19) using loop-mediated isothermal amplification. J. Clin. Virol. 2020;129:104446. doi: 10.1016/j.jcv.2020.104446) can also be utilized. Two tests currently authorized under an emergency use authorization for POC testing include the Accula Rapid PCR Test and Sofia test, among many others.

The Accula test can be processed on-site at a doctor's office or other POC setting and requires a nasal swab from the patient. It can use PCR (NAAT) and lateral flow technology to provide visual detection of the coronavirus SARS-CoV-2 RNA. The test can involve taking a nasal swab; adding the nasal specimens to a SARS-CoV-2 buffer to solubilize the sample; inputting the solution into a test cassette containing positive and negative controls, enzymes, reagents, and a detection strip for the steps in the assay, which include lysis of the virus, reverse transcription of viral RNA to cDNA, nucleic acid amplification and detection. After thirty minutes, the test results can be interpreted by the presence of blue test lines on the detection strip. These results, along with a blue process control line and other information such as a confirmed valid negative test, may be captured by photographic means and further processed to a centralized data center according to embodiments herein. The results can be actually provided in a series of three-letter symbols P, A, or N, with two out of eight combinations of positive indicators. The remaining combinations can be either negative or invalid. Interpretation by an operator and/or through AI means can provide the final interpreted result.

The Sofia test is a SARS antigen test that uses lateral flow immunofluorescent sandwich assays to detect the presence or absence of the nucleocapsid protein antigen from SARS-CoV-2 in nasopharyngeal and nasal swab specimens from individuals potentially exposed to COVID-19. The test can also detect SARS-CoV and does not differentiate between the two viruses. The test can detect various other viruses as would be appreciated by a person of ordinary skill in the art. The results can be displayed on the instrument as positive, negative, or invalid. Similar data from any other testing device, such as but not limited to a POC device, may also be captured according to embodiments. Other POC platforms that are currently approved under emergency authorizations from the Food and Drug Administration (FDA) can include instruments and tests from Abbott, Access Bio, Assure, Azure Bio, Becton Dickenson, Biofire, Capheid, Cue Health, Hangzhou, Lucira, Luminostics, Lumira Dx, MidaSpot, Roche, Salofa Oy, and various other manufacturers as would be appreciated by a person of ordinary skill in the art. Results from any one of these recited instruments may be captured using embodiments herein.

While SARS-CoV-2 is the primary pathogen the world is currently focused on, embodiments here are not limited to POC devices testing for SARS-CoV-2. Other pathogens, which can be tested and for which results can be displayed in capturable or retrievable form include, but are not limited to, influenza and other viral pathogens.

Influenza is caused by an RNA virus of the orthomyxoviridae family. There are three types of these viruses, and they cause three different types of influenza: type A, B, and C. Influenza virus type A viruses infect mammals (e.g., humans, pigs, ferrets, horses, birds, etc.). This type of virus can be very important to mankind, as this is the type of virus that has caused worldwide pandemics.

Influenza A viruses infect a wide variety of mammals, including, but not limited to, humans, horses, pigs, ferrets, birds, etc. Influenza A is one of the main human pathogens, often associated with epidemics and pandemics. There are at least 15 known hemagglutinin (H) serotypes and 9 known neuraminidase (N) serotypes. Pigs and birds are believed to be particularly important reservoirs, generating pools of genetically and antigenically diverse viruses which get transferred back to the human population via close contact between humans and animals.

Influenza B viruses infect mammals only and cause disease, but generally not as severe as influenza A types. Unlike influenza A viruses, influenza B viruses do not have distinguishable serotypes. Influenza C viruses also infect mammals only but rarely cause disease. They are genetically and morphologically distinct from influenza A and B types.

There are 4 antigens present in the influenza virus, the hemagglutinin (HA), neuraminidase (NA), nucleocapsid (NA), the matrix (M), and the nucleocapsid proteins (NP). NP is a type-specific antigen which occurs in 3 forms, A, B, and C, which can provide the basis for the classification of human influenza viruses. The matrix protein (M protein) surrounds the nucleocapsid and makes up approximately 35-45% of the particle mass. Two surface glycoproteins can be seen on the surface as rod-shaped projections. The hemagglutinin (HA) is made up of 2 subunits, HA1 and HA2. HA mediates the attachment of the virus to the cellular receptor. Neuraminidase (NA) molecules are present in lesser quantities in the envelope. Circulating human strains are notorious for their tendency to accumulate mutations from one year to the next and cause recurrent epidemics.

In eukaryotes, sugar residues are commonly linked to four different amino acid residues. These amino acid residues can be classified as O-linked (e.g., serine, threonine, and hydroxylysine) and N-linked (asparagine). The O-linked sugars are synthesized in the Golgi or rough Endoplasmic Reticulum (ER) from nucleotide sugars. The N-linked sugars are synthesized from a common precursor and are subsequently processed. It is known that the addition of N-linked carbohydrate chains is important for stabilization of folding, prevention of degradation in the endoplasmic reticulum, oligomerization, biological activity, and transport of glycoproteins. The addition of N-linked oligosaccharides to specific Asn residues plays an important role in regulating the activity, stability, or antigenicity of mature proteins of viruses (Opdenakker G. et al FASEB Journal 7, 1330-1337 1993). It has also been suggested that N-linked glycosylation is required for folding, transport, cell surface expression, secretion of glycoproteins (Helenius, A., Molecular Biology of the Cell 5, 253-265 1994), protection from proteolytic degradation, and enhancement of glycoprotein solubility (Doms et al., Virology 193, 545-562 1993). Viral surface glycoproteins are not only required for correct protein folding but also provide protection against neutralizing antibodies as a “glycan shield.” As a result, strong host-specific selection is frequently associated with codon positions of potential N-linked glycosylation. Consequently, N-linked glycosylation sites tend to be conserved across strains and clades.

Outbreaks of influenza A virus continue to cause morbidity and mortality worldwide. In the United States alone, an estimated 5 to 20% of the population is infected by influenza A virus annually, causing approximately 200,000 hospitalizations and 36,000 deaths. Currently, there is less reporting about deaths or outbreaks of influenza A due to the overwhelming progression of COVID-19. There is some speculation that mask-wearing around the globe has limited the spread and infection rate of the flu, but embodiments herein, as previously stated, are not limited to testing results for COVID-19. The establishment of comprehensive vaccination policies has been an effective measure to limit influenza morbidity. However, the frequent genetic drifting of the virus requires yearly reformulation of the vaccine, potentially leading to a mismatch between the viral strain present in the vaccine and those circulating. Thus, antiviral therapies against influenza virus can be important tools to limit both disease severity as well as transmission. This can also become important for COVID-19 variants, and future vaccines and vaccination programs may rely upon mixtures of vaccines directed to variants or co-variants.

The highly pathogenic H5N1 influenza viruses have caused outbreaks in poultry and wild birds since 2003 (Li K S et al. (2004) Nature 430:209-213). As of February 2010, these viruses have infected not only avian species but also over 478 humans, of which 286 cases proved to be fatal (www.who.int/csr/disease/avian_influenza/country/cases_table_2010_02_17/en/index .html). The highly pathogenic H5N1 and the 2009 swine-origin influenza A (H1N1) viruses have caused global outbreaks and raised a great concern that further changes in the viruses may occur to bring about a deadly pandemic (Garten R J, et al.(2009) Science 325:197-201, Neumann G, et al. (2009) Nature 459:931-939). There is great concern that an influenza virus would acquire the ability to spread efficiently between humans, thereby becoming a pandemic threat. Therefore, an influenza vaccine can be an integral part of any pandemic preparedness plan.

The virus tested for on the POC devices can thus be selected from COVID-19 and other SARS variants, the influenza virus in all of its forms, and various other viruses or pathogen as would be appreciated by a person of ordinary skill in the art. For example, the virus or pathogen tested can also be selected from the group consisting of respiratory syncytial virus (RSV), chlamydia, adenovirdiae, mastadenovirus, aviadenovirus, herpesviridae, herpes simplex virus 1, herpes simplex virus 2, herpes simplex virus 5, herpes simplex virus 6, leviviridae, levivirus, enterobacteria phase MS2, allolevirus, poxviridae, chordopoxvirinae, parapoxvirus, avipoxvirus, capripoxvirus, leporiipoxvirus, suipoxvirus, molluscipoxvirus, entomopoxvirinae, papovaviridae, polyomavirus, papillomavirus, paramyxoviridae, paramyxovirus, parainfluenza virus 1, mobillivirus, measles virus, rubulavirus, mumps virus, pneumonovirinae, pneumovirus, metapneumovirus, avian pneumovirus, human metapneumovirus, picornaviridae, enterovirus, rhinovirus, hepatovirus, human hepatitis A virus, cardiovirus, andapthovirus, reoviridae, orthoreovirus, orbivirus, rotavirus, cypovirus, fijivirus, phytoreovirus, oryzavirus, retroviridae, mammalian type B retroviruses, mammalian type C retroviruses, avian type C retroviruses, type D retrovirus group, BLV-HTLV retroviruses, lentivirus, human immunodeficiency virus 1, human immunodeficiency virus 2, HTLV-I and -II viruses, SARS coronavirus, herpes simplex virus, Epstein Barr virus, cytomegalovirus, hepatitis virus (HCV, HAV, HBV, HDV, HEV), toxoplasma gondii virus, treponema pallidium virus, human T-lymphotrophic virus, encephalitis virus, West Nile virus, Dengue virus, Varicella Zoster Virus, rubeola, mumps, rubella, spumavirus, flaviviridae, hepatitis C virus, hepadnaviridae, hepatitis B virus, togaviridae, alphavirus sindbis virus, rubivirus, rubella virus, rhabdoviridae, vesiculovirus, lyssavirus, ephemerovirus, cytorhabdovirus, necleorhabdovirus, arenaviridae, arenavirus, lymphocytic choriomeningitis virus, Ippy virus, lassa virus, coronaviridae, coronavirus, torovirus and combinations thereof.

### Example Embodiment

A user may use client device 106 to launch application 112 to upload an image of a diagnostic test result form and POC diagnostic testing media. For example, the user may be associated with an entity performing and/or analyzing POC diagnostic tests. The entity may be a laboratory, hospital, medical facility, etc. The user may launch application 112 to upload an image of a diagnostic test result form and POC diagnostic testing media.

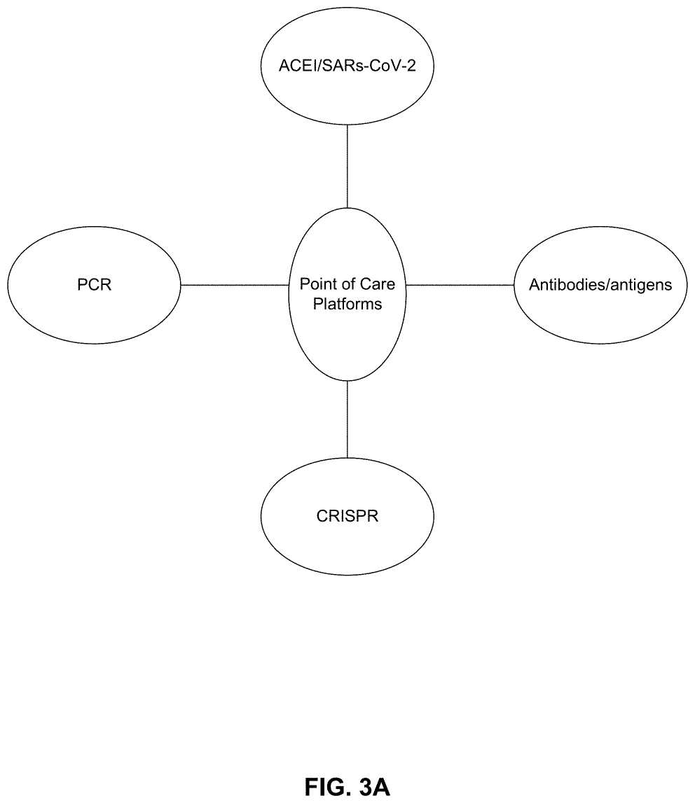

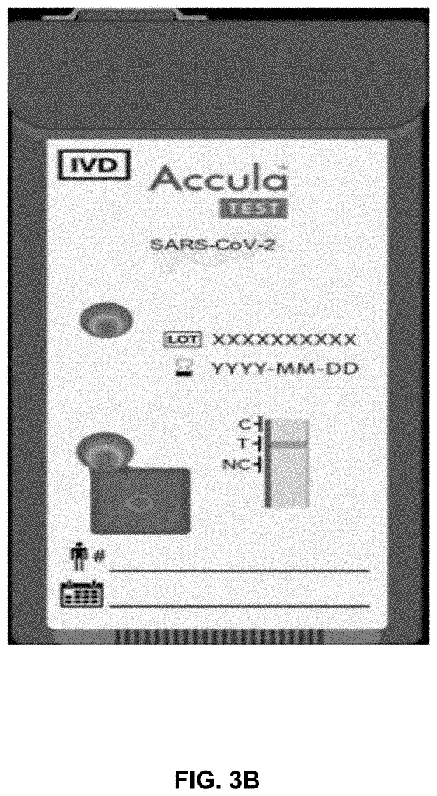

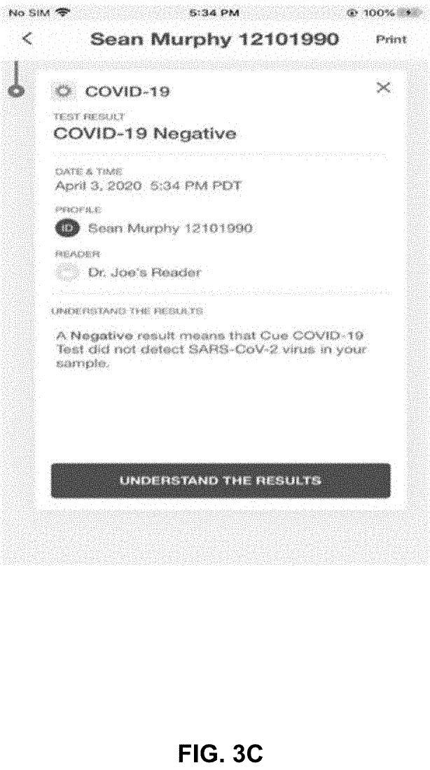

The user or entity may have performed a POC diagnostic test on a patient. As described above, a POC diagnostic test may be a medical test for identifying medical information about a patient. As a non-limiting example, a POC diagnostic testing device may be used to conduct a POC diagnostic test on the patient to detect a particular illness, disease, virus, etc. the POC diagnostic testing device may extract or collect a sample (e.g., salvia, blood, bodily fluid, etc.). The POC diagnostic testing media may provide an analysis of the sample. The POC diagnostic testing media may analyze the sample for presence, absence, or quantity of various attributes associated with the illness, disease, virus, etc. The POC diagnostic testing device and POC testing media will be described in greater detail with respect to FIG. 3.

After conducting the test, the user or entity may fill out a diagnostic test result form based on the POC diagnostic testing media's analysis. The diagnostic test result form may include patient information and attributes that were tested using the POC diagnostic test. The attributes may be associated with the patient. For example, the attributes may correspond with the presence, absence, or quantity of certain elements within the patient. The user or entity may also provide the values for the attributes based on the POC diagnostic test on the diagnostic test result form. For example, the POC diagnostic test may reveal the presence, absence, or quantity of the one or more attributes. The user and entity may provide the indicate the presence, absence, or quantity of the one or more attributes on the diagnostic test result form. As a non-limiting example, to indicate the one or more values on the diagnostic test result form, the user may provide a code, mark a checkbox, fill a bubble, etc., on the diagnostic test. The diagnostic test result form may also include a POC diagnostic test result. The POC diagnostic test result may be interpreted using the one or more values of the one or more attributes and a relationship between the one or more attributes. The diagnostic test result form may be computer generated or handwritten.

The diagnostic test result form may also include a form ID. The form identifier may be an identifier associated with the diagnostic test result form. The form ID may be indicative of a type of POC diagnostic test conducted on the patient.

The POC diagnostic testing media may include the patient's information and specimen information. The specimen information may include an analysis of the patient's sample. For example, the specimen information may include a reaction of the POC diagnostic testing media being in contact with the patient's sample. As a non-limiting example, the POC diagnostic testing media may be made of material that is configured to react based on the presence, absence, or quantity of one or more attributes in the patient's sample.

Application 112 may cause the display of a first graphical user interface (GUI) screen on a display of client device 106. The first GUI may include a prompt to log in to server 102 via application 112. A user may input their user credentials to log in to application 112. Application 112 or server 102 may authenticate the user based on their user credentials. In some embodiments, application 112 may determine the location of client device 106. The location should correspond with the location of where the POC diagnostic test is being performed.

In response to successfully authenticating the user, application 112 may cause the display of a second GUI. The second GUI includes a selection for inputting POC diagnostic test results. In response to the user selecting the selection for inputting the POC diagnostic test results, application 112 may cause camera 110 to become operational.

Camera 110 may continuously capture image frames of objects within the field of view of camera 110. A diagnostic test result form and POC diagnostic testing media may be within the field of view of camera 110. The diagnostic test result form and POC diagnostic testing media may be positioned in a predetermined position. For example, the POC diagnostic testing media may be in the bottom-left corner of the diagnostic test result form and image frame. Furthermore, the diagnostic test result form may include an identifier, such as a barcode or QR code.

Camera 110 may capture an image of the diagnostic test result form and POC diagnostic testing media in response to user input. For example, camera 110 the image based on the user selecting an element on the second GUI. In some embodiments, application 112 may determine that the diagnostic test result form and POC diagnostic testing media are within the field of view of camera 110 and may cause camera 110 to automatically capture the image. Application 112 may transmit the image to server 102.

Server 102 may receive the image of the diagnostic test result form and the POC diagnostic testing media. POC application 108 may identify the POC diagnostic testing media in the image. Furthermore, POC application 108 may confirm that the diagnostic test result form and the POC diagnostic testing media are positioned in the predetermined position such that the patient's information and the patient identifier are visible in the image.

As described above, the diagnostic test form may include patient information (including, but not limited to, the patient identifier, patient name, and accession number), form ID, one or more values, and an interpretation of the test result. The one or more values correspond with one or more attributes of the POC diagnostic test result. For example, the attributes may be associated with attributes that were tested while performing the POC diagnostic test. The values may indicate characteristics about the tested attributes, such as quantity, presentence, absence, level, etc. POC application 108 may extract the patient information (including, but not limited to, the patient identifier, patient name, and accession number) on the diagnostic test result form from the image. The patient information (including, but not limited to, the patient identifier, patient name, and accession number) may include the patient identifier, accession number, patient name, or the like. In some embodiments, POC application 108 may implement optical character recognition (OCR) to extract the patient information (including, but not limited to, the patient identifier, patient name, and accession number)the one or more values, the form ID, and the interpretation of the test result from the diagnostic test result form.

In other embodiments, POC application 108 a machine-learning algorithm to identify and extract the one or more values from the diagnostic test result form. For example, POC application 108 may be trained to identify and extract the one or more values from the diagnostic test result form. POC application 108 may be trained using similar diagnostic test result forms. During the training process, POC application 108 may attempt to identify and extract the one or more values. A user may provide feedback to verify whether POC application 108 accurately identified the one or more values. The feedback may be used to tune POC application 108, such that POC application 108 more accurately identifies the one or more values.

As a non-limiting example, POC application 108 may implement a machine-learning algorithm, such as neural networks (e.g., convolutional neural networks (CNN), recurrent neural networks (RNN), artificial neural networks (ANN), etc.). The machine-learning algorithm may be a supervised or unsupervised algorithm.

Furthermore, POC application 108 may implement a barcode reader to scan the barcode on the diagnostic test result form. The patient identifier may be encoded in the barcode.

POC application 108 may also extract the patient information (including, but not limited to, the patient identifier, patient name, and accession number) on the POC diagnostic testing media from the image using OCR. Furthermore, POC application 108 may implement a barcode reader to scan the barcode on the POC diagnostic testing media. The barcode may be encoded with a unique test-specific code specifying the provider information, the information for the patient, and the specimen information. Furthermore, the barcode may be encoded with the POC diagnostic test result.

POC application 108 may compare the patient information (e.g., patient name and patient identifier) extracted from the diagnostic test result form and the POC diagnostic testing media. POC application 108 may confirm that the diagnostic test result form and the POC diagnostic testing media correspond to the same patient in response to matching the patient information extracted from the diagnostic test result form and the POC diagnostic testing media.

POC application 108 may store the patient information (including, but not limited to, the patient identifier, patient name, and accession number), the form ID, and the one or more values in database 104. POC application 108 may correlate the interpreted POC diagnostic test result with an existing patient record using the patient information (including, but not limited to, the patient identifier, patient name, and accession number). POC application 108 may transmit the patient information (including, but not limited to, the patient identifier, patient name, and accession number), the form ID, and the one or more values to application 112.

Application 112 may receive the patient information (including, but not limited to, the patient identifier, patient name, and accession number), the form ID, and the one or more values from POC application 108. Application 112 may store the patient information, the form ID, and the one or more values in a local storage device on client device 106. The local storage device may be a non-persistent memory on client device 106, such as cache memory.

Application 112 may render a third GUI, which prompts the user to input an accession number and patient's date of birth information. In response to receiving the accession number and the patient's date of birth information, application 112 may attempt to retrieve an order for the POC diagnostic test for the patient using the accession number and the patient's date of birth information. In response to successfully retrieving the order for the POC diagnostic test, application 112 may confirm the identity of the patient by matching the accession number and patient's date of birth information received on the third GUI with the accession number and patient's date of birth information associated with the order.

In response to successfully retrieving the order and verifying the identity of the patient, application 112 may retrieve the form ID and one or more values from the local storage device. Application 112 may determine the type of POC diagnostic test based on the form ID. Furthermore, application 112 may determine the relationship between the attributes tested for the type of POC diagnostic test based on the type of POC diagnostic test.

Application 112 may interpret the diagnostic test result using the one or more values and based on the predetermined relationship between the one or more attributes. For example, application 112 may determine that the one or more values indicate a presence of a first attribute in the patient and an absence of a second attribute in the patient. Application 112 may determine that based on the relationship between the first and the second attribute, and the presence of the first attribute, and the absence of the second attribute, the POC diagnostic test result should be positive.

Furthermore, application 112 may determine whether the values corresponding to the one or more attributes are consistent. For example, application 112 may determine that the presence of the first attribute indicates an absence of the second attribute. As a result, the value corresponding to the second attribute should indicate the absence of the second attribute. In the event application 112 determines that the one or more values indicate that the first attribute and second attribute is present in the patient, application 112 may determine that the diagnostic test result form has erroneously marked either the presence of the first attribute or the second attribute.

In some embodiments, the extracted interpretation of the test result may be a POC diagnostic test result (e.g., positive, negative, inconclusive, etc.), indicated on the diagnostic test result form by the user or entity performing the POC diagnostic test. POC application 108 may transmit the extracted interpretation of the POC diagnostic test result to application 112.

Application 112 may compare the interpretation of the POC diagnostic test result generated by application 112 with the interpretation of the POC diagnostic test result extracted from the diagnostic test result form to verify that the interpretation of the POC diagnostic test result on the diagnostic form is accurate. In the event that the interpretation of the POC diagnostic test result generated by application 112 matches the interpretation of the POC diagnostic test result extracted from the diagnostic test result form, application 112 may output the interpreted POC diagnostic test result.

Alternatively, in the event that the interpretation of the POC diagnostic test result generated by POC application 108 does not match the interpretation of the POC diagnostic test result extracted from the diagnostic test result form, POC application 108 may cause the display of a message on a GUI of application 112 indicating the discrepancy between the interpretation of the POC diagnostic test result generated by POC application 108 and the interpretation of the POC diagnostic test result extracted from the diagnostic test result form. A user may provide input to confirm the interpretation of the POC diagnostic test result.

A patient may access the POC diagnostic test result using a patient portal 114 using user device 107. For example, the patient may access patient portal 114 by launching patient portal 114 application or navigating to patient portal 114 using an Internet browser. Patient portal 114 may transmit a request to server 102 to retrieve a POC test result. The request may include the patient information (e.g., accession number, patient identifier, patient name, etc.). POC application 108 may retrieve the POC diagnostic test result from database 104 using the patient information and cause display of the POC diagnostic test result on patient portal 114.

In some embodiments, POC application 108 may identify and extract specimen information on the POC diagnostic testing media from the image. For example, the specimen information may be a single horizontal line. POC application 108 may use the machine-learning algorithm to identify and extract the single horizontal line from the image. POC application 108 may transmit the specimen information to application 112. Application 112 may determine whether the one or more values are consistent with the specimen information based on a predetermined set of rules.

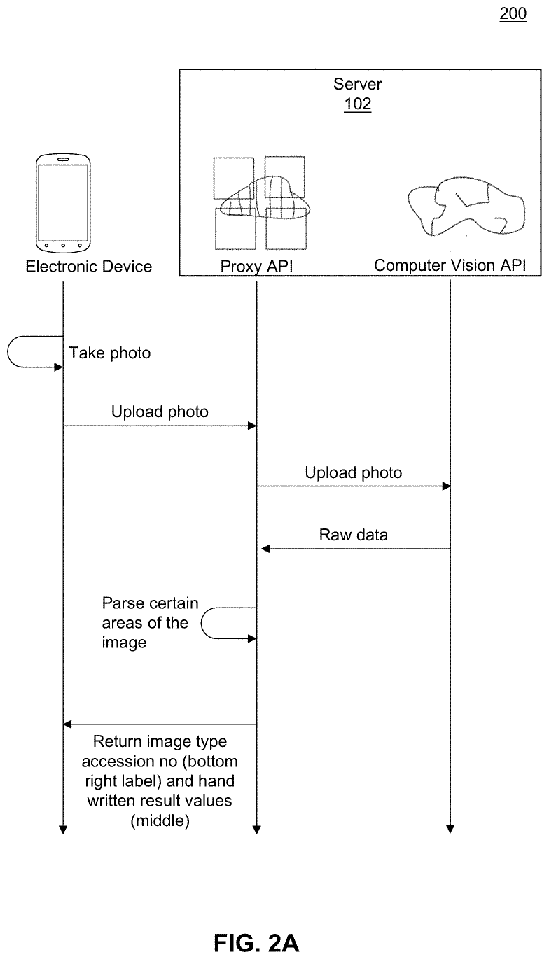

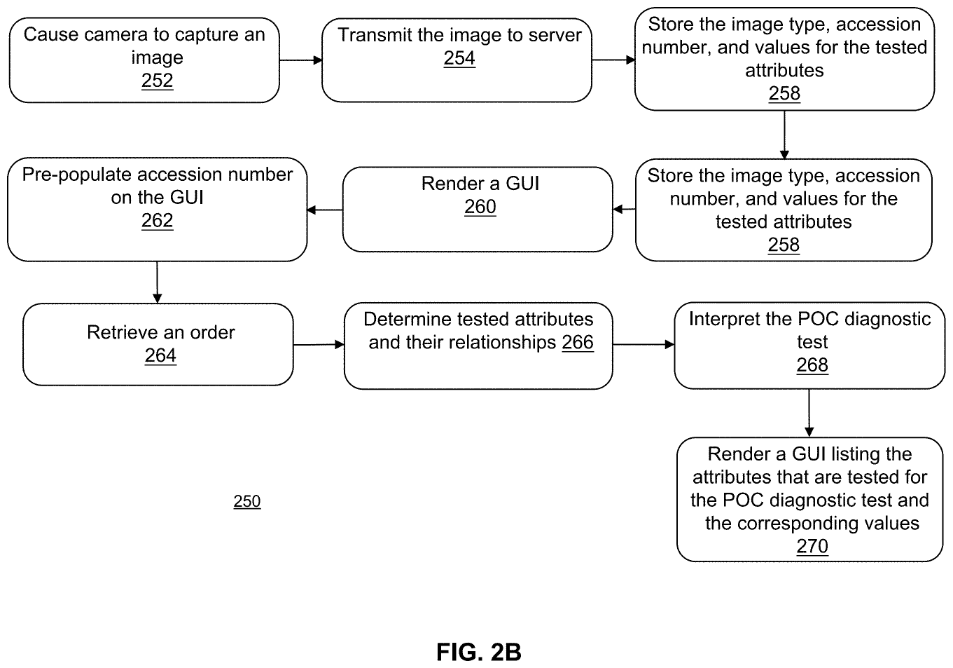

FIG. 2A is a flowchart illustrating method 200 for identifying and extracting information on a diagnostic test result form and POC diagnostic testing media, according to some embodiments.

Method 200 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously or in a different order than shown in FIG. 2, as will be understood by a person of ordinary skill in the art. Method 200 shall be described with reference to FIG. 1. However, method 200 is not limited to those example embodiments.

In 202, server 102 receives an image of a diagnostic test result form and a POC diagnostic testing media from application 112 of client device 106. Specifically, server 102 may receive the image according to API 220. The diagnostic test result form and the POC diagnostic testing media are positioned in the predetermined position such that the patient's information and the patient identifier are visible in the image. The diagnostic test result form may include a POC diagnostic test result of a POC diagnostic test conducted on a patient, patient information, and values of attributes tested with the POC diagnostic test. The patient information may include an accession number. Furthermore, the diagnostic test result form may include a form ID. The form ID may indicate an image type.

In 204, server 102 forwards the image to POC application 108. POC application 108 may implement an OCR engine and a machine-learning algorithm to identify and extract information from the diagnostic test result form and the POC diagnostic testing media. For example, POC application 108 may implement AZURE Computer Vision (developed by MICROSOFT) and AZURE Form Recognizer (developed by MICROSOFT).

POC application 108 may identify the text on the diagnostic test result form and the POC diagnostic testing media. The text may include a POC diagnostic test result of a POC diagnostic test conducted on a patient, patient information, values of attributes tested with the POC diagnostic test, and form ID. The POC diagnostic testing media may include the patient information. For example, POC application 108 may use the OCR engine to identify and extract the form ID and patient information. The machine-learning algorithm may identify and extract values corresponding to the attributes tested using the POC diagnostic test.

As a non-limiting example, the one or more attributes may be text, and the values corresponding to each of the one or more attributes may be indicated using checkboxes. Specifically, one or more checkboxes may correspond to each attribute. Each checkbox may correspond with a different value. A user or entity conducting the POC diagnostic test may mark the checkbox corresponding with the value for the attribute that was tested. The machine-learning algorithm may be trained to identify which checkbox is marked for each respective attribute. Based on identifying which checkbox is marked, the machine-learning algorithm may identify the value corresponding to the marked checkbox. The machine-learning algorithm may correlate the value to the respective attribute.

In 206, POC application 108 transmits the identified and extracted information from the diagnostic test result form and the POC diagnostic testing media to API 220. The information may be in the form of raw data.

In 208, server 102 parses certain areas of the image.

In 210, server 102 transmits the image type, accession number, and values for the tested attributes that were identified and extracted from the diagnostic test result form and POC diagnostic testing media to application 112.

FIG. 2B is a flowchart illustrating method 250 for outputting the POC diagnostic test result, according to some embodiments.

Method 250 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously or in a different order than shown in FIG. 2B, as will be understood by a person of ordinary skill in the art. Method 250 shall be described with reference to FIG. 1. However, method 250 is not limited to those example embodiments.

In 252, application 112 causes camera 110 to capture an image of a diagnostic test result form and POC diagnostic testing media based on user input. The diagnostic test result form may include a POC diagnostic test result of a POC diagnostic test conducted on a patient, patient information, and values of attributes tested with the POC diagnostic test. The patient information may include an accession number. Furthermore, the diagnostic test result form may include a form ID. The form ID may indicate an image type.

In 254, application 112 transmits the image to server 102 to identify and extract information from the diagnostic test result form and POC diagnostic testing media.

In 256, application 112 receives the image type, accession number, and values for the tested attributes from server 102. The image type, accession number, and values for the tested attributes may be identified and extracted as described in method 200.

In 258, application 112 stores the image type, accession number, and values for the tested attributes in a session or on a local storage device.

In 260, application 112 renders a GUI, which prompts the user to input the accession number and the month and day of the patient's date of birth, in response to storing, the image type, accession number, and POC diagnostic test result.

In 262, application 112 pre-populates the accession number on the GUI. For example, the GUI may include text input boxes for the accession number and the month and day of the patient's date of birth. Application 112 may retrieve the stored accession number and pre-populate the accession number in the text input box. The user may input the month and day of the patient's date of birth. The user may select the confirm button on the GUI to confirm the accession number and the month and day of the patient's date of birth.

In 264, application 112 retrieves an order using the month and day of the patient's date of birth and accession number from the entity that conducted the POC diagnostic test. For example, application 112 may transmit a request to server 102 to retrieve the order of the POC diagnostic test corresponding to the patient. POC application 108 may forward the request to the entity that conducted the POC diagnostic test (e.g., from Insight DX). The entity may return the order corresponding to the patient's date of birth and accession number. POC application 108 may transmit the order to application 112. The order may be an order code that identifies the POC diagnostic test that was conducted on the patient. Application 112 may identify the POC diagnostic test based on the order code.

In 266, application 112 determines the attributes that are tested for the identified POC diagnostic test and the relationship between the attributes. Application 112 may determine the attributes that are tested for the identified POC diagnostic test and the relationships based on predetermined rules regarding the POC diagnostic test. Application 112 may also retrieve the stored values of the tested attributes and correlate the stored values to the respective attributes.

In 268, application 112 interprets the POC diagnostic test result based on the values corresponding to the respective attributes and the relationship between the attributes. As a non-limiting example, the POC diagnostic test result may be “positive” or “negative.”

In 270, application 112 renders a GUI listing the attributes that are tested for the POC diagnostic test and the corresponding values. Each value may be listed in an input box, such as a dropdown. As such, the user may modify the value using the input box as necessary. Furthermore, in the event server 102 is unable to identify and extract a given value from the diagnostic test result form, the input box for the corresponding attribute may be empty. The interpretation of the POC diagnostic test result may also be included on the GUI. In response to changing the value, application 112 may update the interpretation of the POC diagnostic test result. The GUI may also include the accession number and the date and month of the patient's date of birth. The user may select a confirm button to confirm the accuracy of the values and the interpretation of the POC diagnostic test result. In some embodiments, the GUI may provide a visual indicator based on the interpretation of the POC diagnostic test result. For example, for an interpretation of the POC diagnostic test result that is “positive,” application 112 may cause the GUI's background to be red. Alternatively, for an interpretation of the POC diagnostic test result that is “negative,” application 112 may cause the GUI's background to be green. Furthermore, for an interpretation of the POC diagnostic test result that is “invalid” (inconclusive), application 112 may cause the GUI's background to be grey.

As noted previously, if the particular test and instrument provides results that do not need to be interpreted or reprocessed, the image is simply captured directly with the results posted. In addition, if raw data is presented in the form of lines, or a combination of the presence or absence of line or colored line combinations, the image captured may be such direct line presentation and the algorithm and/or software on or used with the electronic device can “interpret” the raw presentation and deliver the final verified results as positive, negative, or invalid. Similarly, an operator or reader may further interpret the colored line or line combinations and present a capturable image as letter combinations or code for the results, which the user photographs and further processes through the app into the final verified results.

FIG. 3A illustrates a diagram of the various technologies used in the POC platforms currently authorized by the FDA under emergency use authorization, according to some embodiments. As described above, and as shown in FIG. 3, rt PCR can be utilized to amplify nucleotides in the virus in addition to tests that test for the presence of either antibodies or antigens generated by exposure to COVID-19 or present as a protein or protein fragment in the virus.

Table 2 describes various assays, technologies, and generated results that can
be captured by an electronic device (e.g., smart phone) with its camera.

| Company/Device | Technical Basis of Test | Results Display |
|---|---|---|
| Abbott/Binax Now | Nucleocapsid antigen/lateral | Negative Result-one pink |
| | flow immunoassay | purple control line. Positive |
| | | result-two pink/purple control |
| | | lines; Invalid result: No |
| | | control line, sample line only; |
| | | blue control line only; one |
| | | blue control line and one |
| | | sample line. |
| Access Bio/CareStart | Nucleocapsid protein | Control red, Test |
| Rapid POC diagnostic | antigen/lateral flow | blue = positive |
| test | assay/immunochromatographic | Control red, test |
| | | absent = negative |
| | | Test blue, control |
| | | absent or control |
| | | and test absent-invalid |
| Mesa Biotech/Accula or | PCR/lateral flow for SARS- | C = Internal PositiveProcess |
| Silaris Dock, as shown in | CoV-2 RNA/nucleic acid | control |
| FIG. 3B. | amplification test (NAAT) | T = SARS-CoV-2 |
| | | NC = Internal Negative |
| | | Process Control. C blue; T |
| | | blue; NC blank = positive; C |
| | | blank, T blue, NC |
| | | blank = positive; T blue; NC |
| | | blank, C blank = positive. |
| | | C blue; T and NC |
| | | blank = negative |
| | | C, T, NC blue; C, T blank, |
| | | NC blue; C, T, NC |
| | | blank = invalid |
| Assure RapidTest | Immunoglobulin M | C, IgM, and IgG. Control line |
| Azure | (IgM)/Immunoglobulin | changes from blue to red; |
| Biosystems/AssureUS | G (IgG) assay/adaptive | colored lines appear in IgG or |
| distributor | immune response assay | IgM regions. C red, IgG red; |
| | for exposure toCOVID-19 | IGM red (IgM, IgG positive) |
| | recent or prior infection | or C red, IgG red, IgM blank |
| | (COVID-19 virus- | (IgG positive); C red, IgG |
| | specificantibodies) | blank; IgM red (IgM |
| | | positive). C red, IgG, and |
| | | IgM blank-negative. Other |
| | | combinations are invalid. |
| Adial Assure distribitor | IgG/IgM rapid test | Same as above |
| Becton Dickenson/BD | | Displays digital CoV2: + or − |
| Veritor Plus | | |
| Biofire EZ | Multiplex PCR test for | SARs CoV2 detected or not |
| | SARSCoV2-1 spike protein | detected as shown on test |
| | (S) and SARS CoV2-2 | report. |
| | membrane (M) protein | |
| Cepheid Xpert Xpress | RT-PCR | Results displayed as positive |
| | | or negative on test results |
| | | screen |
| Cue Health/uses Cue | Nucleic acid amplification | Test results displayed on |
| Health Mobile | | phone, as shown in FIG. 3C. |
| application | | |
| Hangzhou LYHER | IgM/IgG colloidal | C, IgM and IgG. Control line |
| | gold/immobilized SARS-CoV- | changes from blue to red; |
| | 2 spike protein and mouse IgG | colored lines appear in IgG or |
| | labeled antibody. | IgM regions. C red, IgG red; |
| | | IGM red (IgM, IgG positive) |
| | | or C red, IgG red, IgM blank |
| | | (IgG positive); C red, IgG |
| | | blank; IgM red (IgM |
| | | positive). C red, IgG and IgM |
| | | blank-negative. Other |
| | | combinations invalid. |
| Lucira Home | | Positive or negative result |
| | | displayed on the test base. An |
| | | invalid test is shown if both |
| | | positive and negative are |
| | | displayed. |
| Luminostics Clip Rapid | Nucleocapsid protein antigen | Results displayed as positive, |
| Antigen test | detection | negative or invalid on mobile |
| | | device. |
| Lumira Dx UK | Nucleocapsid protein antigen | Results displayed as positive |
| | detection. Test strip using | or negative. |
| | microfluidic | |
| | immunofluorescence. | |
| Midaspot antibody | | |
| combo | | |
| Quidel QuickVue | Nucleocapsid protein | Any shade of pink to red test |
| | antigen/lateral flow | line and the appearance of a |
| | immunoassay | blue procedural control line is |
| | | positive result for SARS |
| | | antigen. Appearance of blue |
| | | procedural control line is |
| | | negative test (needs |
| | | confirmation with molecular |
| | | assay). Blue line not |
| | | appearing for control even if |
| | | pink to red test line appears is |
| | | invalid test. |
| Roche | Rapid chromatographic | Displays colored bands; test |
| | immunoassay for | line and control line show is |
| | nucleocapside protein antigen | positive; control line only- |
| | | negative; test line and no |
| | | control line or no lines- |
| | | invalid. |
| Salofa Oy/Salocor | IgG/IgM rapid test. | Results displayed on card |
| Sofia | Nucleocapsid protein | Positive/Negative or Invalid |
| | antigen/Immunofluorescent | |
| | sandwich assay | |

Table 2 is non-limiting, and the plurality of tests and POC devices includes any test developed and which has an image in any form displayed on the device or reader associated with the device. Some of the devices may have results which are directly captured without the need for an image captured by the app, but embodiments herein include combinations of patients and/or group data that includes results from both and which are presented to the healthcare provider and stakeholders, including the patients. In addition, the same patient may use, over a period of time, POC devices from multiple vendors, which can involve data input from each testing source to provide the complete historical set in the most current testing results.

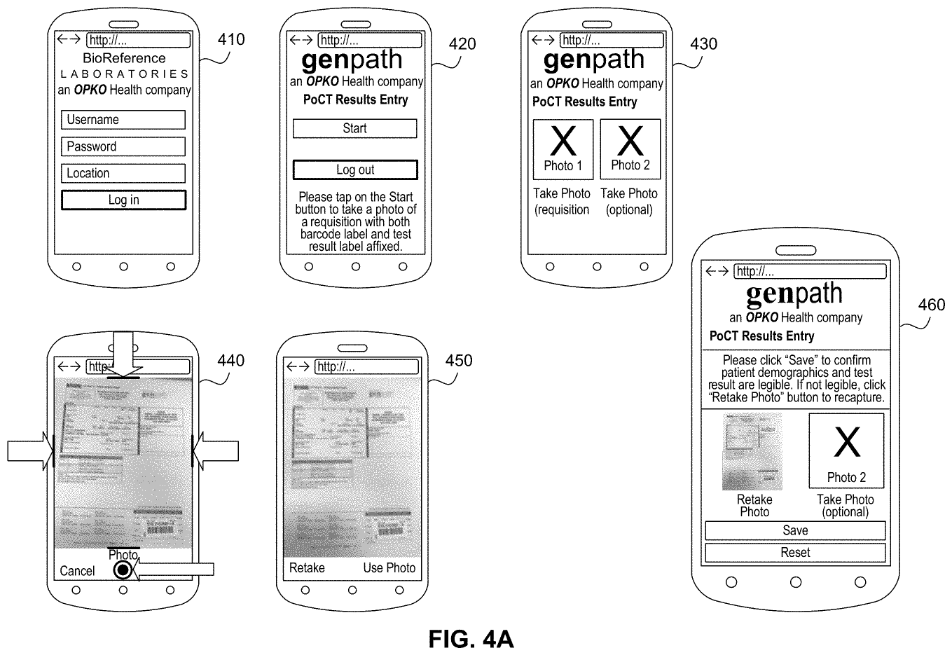

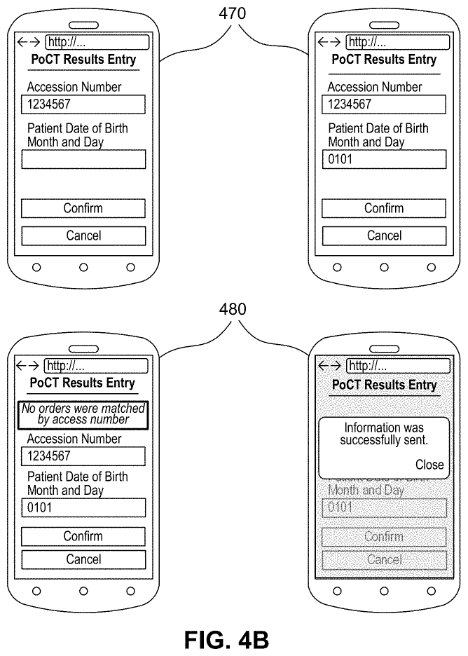

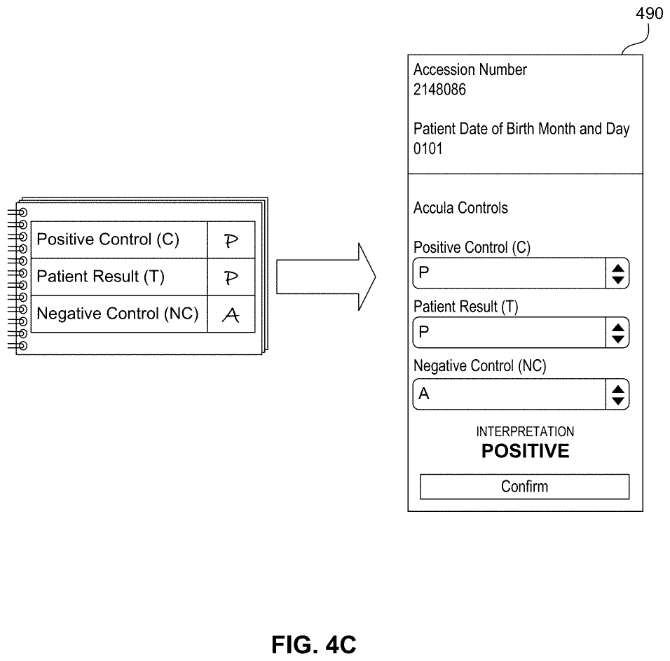

FIGS. 4A, 4B and 4C illustrate graphical user interfaces (GUI) of application 112, according to some embodiments. FIGS. 4A-4C shall be described with reference to FIG. 1, however, are not limited to those example embodiments.

A user may launch application 112 on client device 106 by, for example, by selecting an icon for application 112. Alternatively, the user may search for application 112 through their web browser (e.g., by going to the URL: https://brli.insightdx.com/sofialogin).

In response to launching application 112, application 112 may render a main log-in page 410. Main log-in page 410 may include a GUI. The GUI may include input fields for inputting authentication credentials, such as username, password, and location. As indicated above, the location may be the same location as where a POC diagnostic test was conducted. For example, the location may be a laboratory, medical facility, medical provider location, drive-through test facility, etc.

The user may input their username, password, and PSC location account number in the corresponding input fields. For example, the user may input this information like they would enter it to access the InsightDx application or patient portal. Insight DX is a proprietary system for entering patient-specific information to access testing services and receive results generated from the tests conducted. It can permits and facilitate the electronic transmission of data between physicians or other health care providers and laboratories and include test results, claims eligibility documents, claims to process documents and medical data. Once the user profile is authenticated, application 112 may load a start page 420. The user may select a ‘Start’ button on start page 420 when they have a requisition ready to be captured. The requisition may be the diagnostic test result form and the POC diagnostic testing media.

In response to the selection of the ‘Start’ button on start page 420, application 112 may render an upload photo page 430. Upload photo page 430 may include selections to “Take Photo” for capturing an image of the requisition. The user may select “Take Photo” when the user is ready to upload an image of the requisition.

In response to selecting the “Take Photo” button, application 112 may cause camera 110 of client device 106 to become operational. Application 112 may cause the field of view 440 of camera 110 to be rendered. The user may confirm that they placed the requisition on a well-lit, flat surface before taking the picture. The user may line up the requisition in the camera 110's field of view, making sure that all content is in focus and is not cut off on any edge. Field of view 440 may include markers to indicate the edges of the image to be captured. Application 112 may cause camera 110 to capture the image of the requisition based on the user's input. For example, the user may select an option to capture the image or select a button on client device 106.

Once the user has taken the picture, application 112 may render a captured image 450 of the requisition. The user is given the opportunity to review the captured image 450 to confirm that it is focused and none of the content is cut off If the user is not satisfied with captured image 450, the user can select “Retake.” In response to the user selecting “Retake”, application 112 may cause camera 110 to become operational. If the image is acceptable, the user may select “Use Photo.”

In response to the user selecting “Use Photo”, application 112 may render an updated upload photo page 460. Updated upload photo page 460 includes a preview of the captured image as it will appear downstream to the lab users. Updated upload photo page 460 may include selections to save, reset the captured image, retake photo, and take a second photo. The user may select “Save” to save the captured image. Alternatively, the user may select “retake photo” to retake the image. In response to the user selecting “retake photo”, application 112 may cause camera 110 to become operational. Furthermore, the user may select “take a second photo” if they have a 2nd image to upload for the same accession number (e.g., a separate result page). In some embodiments, the accession barcode must be added to the bottom right corner of the second image as well.

Once the image is saved, the image may be transmitted to server 102. Server 102 may identify and extract information from the image, as described in method 200 in FIG. 2A and method 250 in FIG. 2B. Server 102 may return the extracted accession number, image type, and values corresponding to the attributes tested using the POC diagnostic test.

With reference to FIG. 4B, in response to the user selecting “Save”, application 112 may render accession number and date of birth page 470. Accession number and date of birth page 470 may include input fields for inputting the accession number and day and month of the patient's date of birth. The accession number may be pre-populated in the input field corresponding to the accession number, as described in method 200 in FIG. 2A and method 250 in FIG. 2B. The user may input the day and month of the patient's date of birth in the respective field (e.g., in the format MMDD). Once the accession number and the day and month of the patient's date of birth have been input, the user may select “Confirm.”

In response to the user selecting confirm, application 112 may confirm whether the accession number and the day and month of the patient's date of birth matches the accession number and the day and month of the patient's date of birth extracted from the image, as received from server 102, as described in method 200 in FIG. 2A and method 250 in FIG. 2B.

Application 112 may attempt to identify orders of POC diagnostic tests based on the accession number and the day and month of the patient's date of birth. In response to successfully or failing to confirm that order exists corresponding to the accession number and the day and month of the patient's date of birth, application 112 may render success or failure page 480. If application 112 successfully finds an order, success, or failure, page 480 may display a message indicating that the information has been transmitted. If application 112 fails to find an order, success or failure page 580 may display a message indicating an order was not found.

In some embodiments, application 112 may receive and transmit the test results to server 100. In some other embodiments, a diagnostic testing device may interface with a middleware application to transmit the test results to server 100. As non-limiting examples, a diagnostic testing devices such as, but not limited to, TJ18 SOFIA and CEPHEID may interface with a middleware application to transmit test results to server 100. Furthermore, diagnostic testing devices such as, but not limited to, TK77 Accula or TK90 BinaxNOW, BD Veritor, Visby, BinaxNOW, *LumiraDx, and CareStart may use application 112 to transmit the test results. If the test is TJ18 SOFIA, or if the location entered at login is not enabled for the TK77 Accula or TK90 BinaxNOW workflow, the image is sent after accession number and DOB values are validated to match.

With reference to FIG. 4C, in response to application 112 successfully finding an order corresponding to the accession number and the day and month of the patient's date of birth, application 112 may render POC diagnostic test result page 490. POC diagnostic test result page 490 may include values corresponding to the attributes tested with the POC diagnostic test. Application 112 may populate POC diagnostic test result page 490 with the values of the attributes tested with the POC diagnostic test as described in method 200 of FIG. 2A and method 250 of FIG. 2B. Each value may be populated in a dropdown field. As such, the user may change the value as necessary. For example, “A” may indicate an absent attribute, and “P” may indicate a present attribute. Furthermore, as indicated above, certain fields may be blank in the event server 102 was unable to extract a given value. Application 112 may interpret the POC diagnostic test result based on the values of the attributes as described above with respect to method 200 of FIG. 2A and method 250 of FIG. 2B. POC diagnostic test result page 490 may include the interpreted POC diagnostic test result. The user may confirm the values and the interpreted POC diagnostic test result and select “Confirm” on POC diagnostic test result page 490.

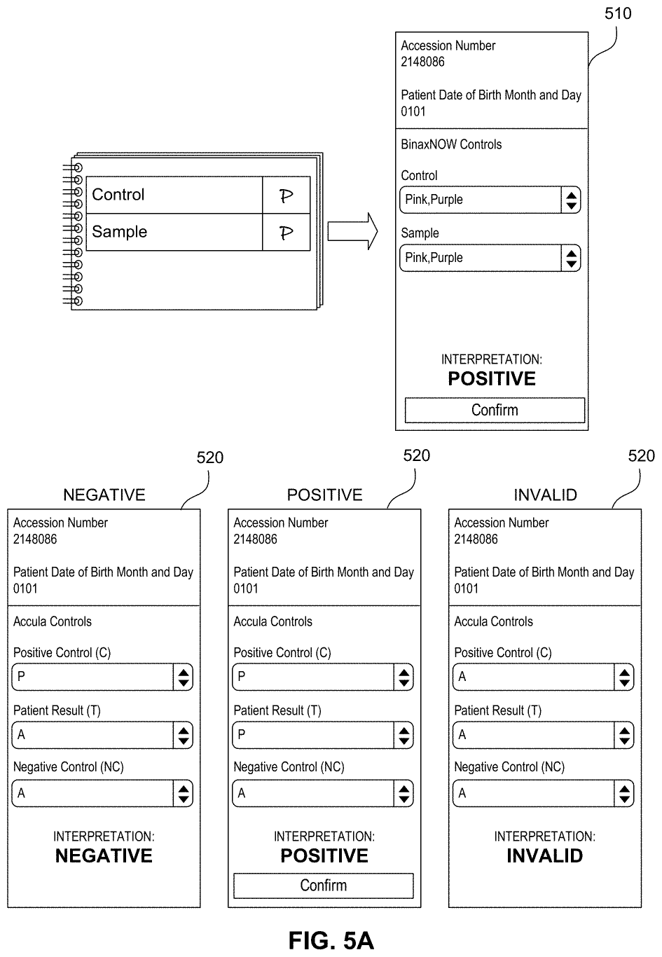

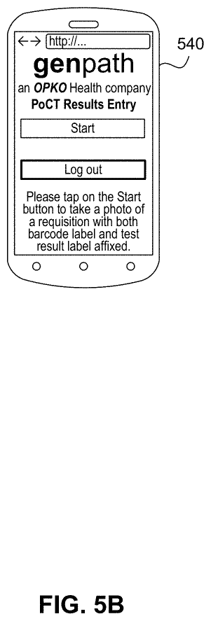

FIGS. 5A and 5B illustrate GUIs of application 112, according to some embodiments.

In some embodiments, application 112 may render POC diagnostic test result page 510. POC diagnostic test result page 510 may include values corresponding to the attributes tested with the POC diagnostic test. Application 112 may populate POC diagnostic test result page 510 with the values of the attributes tested with the POC diagnostic test as described in method 200 of FIG. 2A and method 250 of FIG. 2B. Each value may be populated in a dropdown field. As such, the user may change the value as necessary. For example, “A” for Absent or “B” for Blue or “P” for Pink/Purple. Application 112 may interpret the POC diagnostic test result based on the values of the attributes as described above with respect to method 200 of FIG. 2A and method 250 of FIG. 2B. POC diagnostic test result page 510 may include the interpreted POC diagnostic test result. The user may confirm the values and the interpreted POC diagnostic test result and select “Confirm” on POC diagnostic test result page 510.

Application 112 may also render POC diagnostic test result page 520, which may include a visual indicator corresponding to the interpretation of the POC diagnostic test result. The visual indicator may be a different color background, animation, patterns, shading, etc. For example, the background POC diagnostic test result page 520 may be a green background for when the interpretation of the POC diagnostic test result is negative. Furthermore, the background of POC diagnostic test result page 520 may be red when the interpretation of the POC diagnostic test result is positive. Additionally, the background of POC diagnostic test result page 520 may be red when the interpretation of the POC diagnostic test result is invalid or inconclusive. As a non-limiting example, the interpretation of the POC diagnostic test result may be invalid or inconclusive if more than a threshold number of attributes have a value of absent. Alternatively, the interpretation of the POC diagnostic test result may be invalid or inconclusive if the values of the attributes are inconsistent with each other based on the relationship between the attributes.

In response to the user selecting confirm on POC diagnostic test result page 510 or 520, application may navigate back to start page 420, as shown in FIG. 4. The user can select “Start” to repeat the process on the next requisition or “Log out” to close out of the application. In some embodiments, if application 112 may detect inactivity for a period of time (e.g., 15 minutes), application 112 may automatically log out the operator.

In some embodiments, high throughput data flow of information collected from disconnected Point-of-Care testing platforms having results from COVID-19 or other viral/microbial testing of a subject comprises the steps of (1) optionally pre-registering for the test, booking appointments and securing relevant clinical information; (2) registration to capture and/or confirm subject demographics and order information for the test; (3) preparation; printing/verifying specimen labels, testing form; (3) specimen collection from the subject; (4) testing, preparing “cassette” having antibody/antigen reagents or other components (probes, primers, amplification reagents) depending upon the test and running the assay; (5) capture results of test/testing form using app (AI capable) or digital/photographic means; (6) send the data to capture system (Laboratory Management System) and (7) provide results to need-to-know stakeholders including EMR, Patient portals via FHIR APIs and provided to public health agencies. In an embodiment, the portable devices, the POC devices, are not digitally linked but, rather, are selected from a group of disconnected devices testing patients that are part of a group or sub-group such as a sports team or cruise passenger.

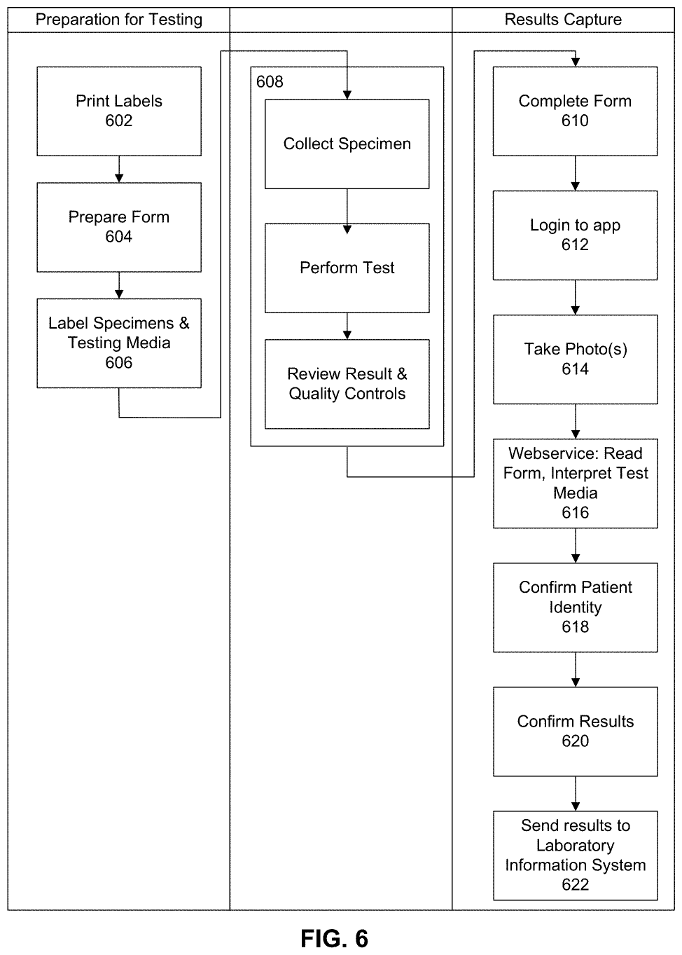

FIG. 6 is a flowchart illustrating method 600 for preparing for testing through sending the results of the POC diagnostic test results to the Laboratory Information System (LIS), according to some embodiments. It is to be appreciated that not all steps may be needed to perform the disclosure provided herein. Further, some of the steps may be performed simultaneously or in a different order than shown in FIG. 6, as will be understood by a person of ordinary skill in the art.

Method 600 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously or in a different order than shown in FIG. 6, as will be understood by a person of ordinary skill in the art. Method 600 shall be described with reference to FIG. 1, however, are not limited to those example embodiments

In 602, barcoded labels are printed using a software application. These labels can contain human-readable text containing provider, patient, and specimen information. There can be a unique identifier for the specimen printed in human-readable text and encoded in the barcode.

In 604, a diagnostic test result form is prepared with information on the provider, patient, and specimen. The diagnostic test result form can later be used to capture the test results, and an image can be taken for automated data extraction.

In 606, the labels of 702 are affixed to the specimen container and POC diagnostic testing media.

In 608, the specimen is collected, the test performed, the results reviewed, and quality controls performed. These processes can follow the protocols dictated by the instrument manufacturer, performing laboratory, and relevant regulatory agencies.

In 610, after testing is complete and the results have been verified, a medical technologist captures the test/QC results on a form and places the POC diagnostic testing media in the predetermined position.

In 612, a user (e.g., the medical technologist) may interact with client device 106 to launch application 112 and uses their credentials to log in.

In 614, the user uses application 112 to take a photo of the form and testing media as described in method 200 of FIG. 2A and method 250 of FIG. 2B.

In 616, application 112 transmits a request to server 102 to extract data from the diagnostic test result form and POC diagnostic testing media, as described in method 200 of FIG. 2A and method 250 of FIG. 2B. Server 102 returns the extracted data, including, but not limited to, provider, patient, specimen, and results information, to application 112.

In 618, the user uses application 112 to enter in snippets of the patient and/or specimen identifiers to ensure proper patient identity.

In 620, the user uses application 112 to review the Quality Control and Test Results extracted from the form to ensure they are accurate.

In 622, after results have been confirmed, the results are automatically transmitted to the Laboratory Information System for results reporting to the stakeholders.

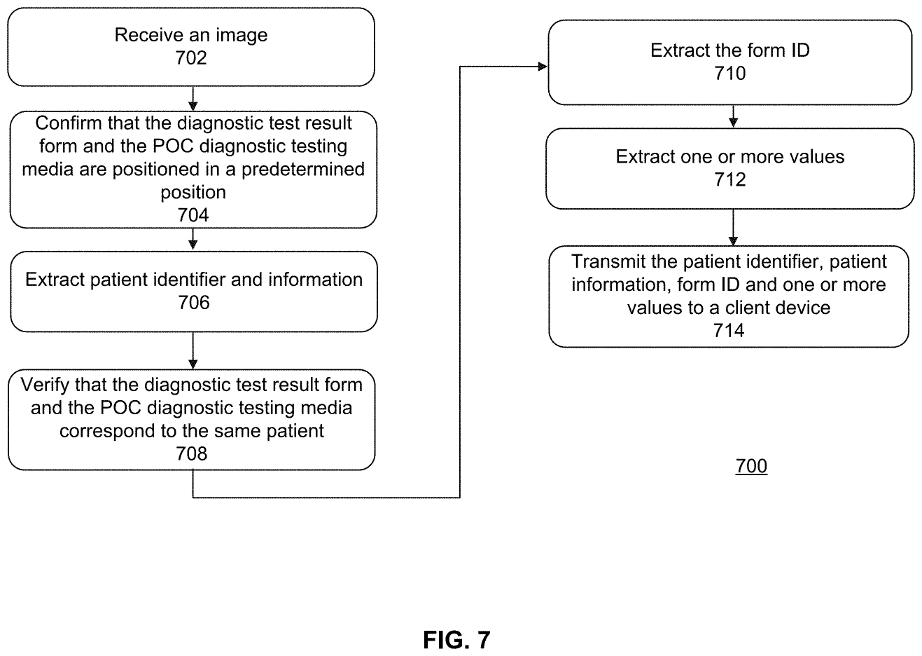

FIG. 7 is a flowchart illustrating a method 700 for linking a POC testing media and a diagnostic test result form, according to some embodiments. This method for integrating an offline POC diagnostic device with a laboratory information system using a client device solves the technological problems related to order integration, automatic barcode reading capability, and results integration.

Method 700 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously or in a different order than shown in FIG. 7, as will be understood by a person of ordinary skill in the art. Method 700 shall be described with reference to FIG. 1, however, are not limited to those example embodiments.

In 702, server 102 receives an image of the diagnostic test result form and a POC diagnostic testing media. The diagnostic test result form includes a POC diagnostic test result generated based on a POC diagnostic test being performed on a patient using a POC diagnostic testing device, a form ID, and a patient identifier. The POC diagnostic testing media includes the patient's information and specimen information.

In 704, server 102 confirms that the diagnostic test result form and the POC diagnostic testing media are positioned in a predetermined position such that the patient's information and the patient identifier are visible in the image.

In 706, server 102 extracts the patient identifier from the diagnostic test result form and the patient information from the POC diagnostic testing media. Server 102 may use OCR to extract the patient identifier and the patient information.

In 708, server 102 verifies that the diagnostic test result form and the POC diagnostic testing media correspond to the same patient. Server 102 may use the patient identifier and patient information to verify that the diagnostic test result form and the POC diagnostic testing media.

In 710, server 102 extracts the form ID on the diagnostic test result form from the image. Server 102 may use OCR to extract the form ID. The form ID may be indicative of a type of POC diagnostic test.

In 712, server 102 extracts one or more values on the diagnostic test result form from the image. The one or more values correspond with one or more attributes of the POC diagnostic test result. The one or more attributes may be tested using the POC diagnostic test. The one or more values may be used to interpret the POC diagnostic test result. Server 102 may use a machine-learning algorithm to extract the one or more values.

In 714, server 102 transmits the patient information, the patient identifier, the form ID, and the one or more values to client device 106. Client device 106 may output the patient information, the patient identifier, the form ID, and the one or more values. Client device 106 may also interpret the POC diagnostic test result based on the one or more values.

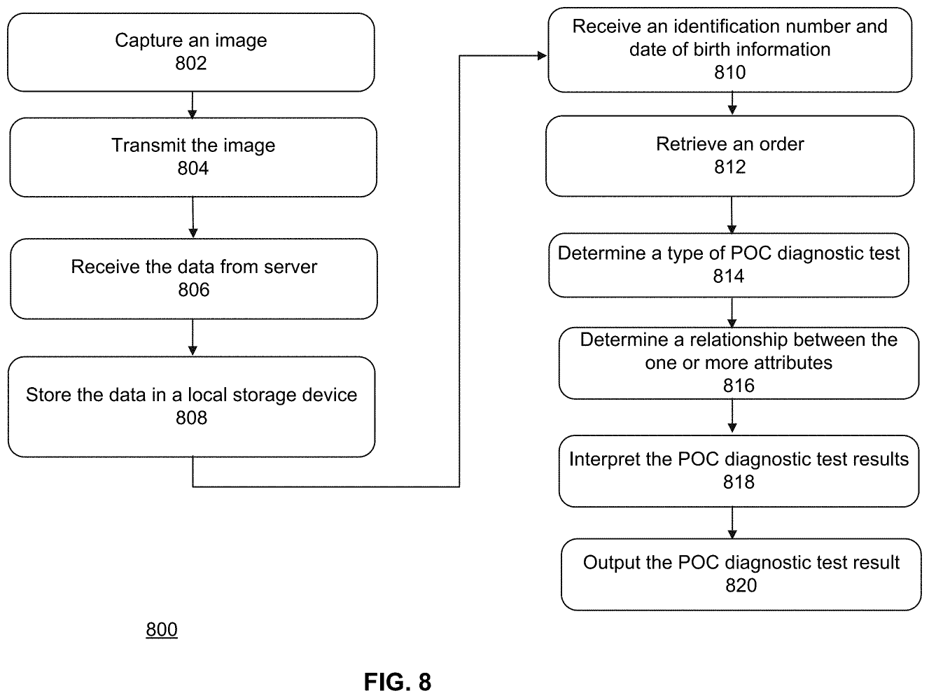

FIG. 8 is a flowchart illustrating a method 800 for interpreting a POC diagnostic test result, according to some embodiments. Method 800 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously or in a different order than shown in FIG. 8, as will be understood by a person of ordinary skill in the art. Method 800 shall be described with reference to FIG. 1, however, are not limited to those example embodiments.

In 802, application 112 of client device 106 causes camera 110 to capture an image of a diagnostic test result form and a POC diagnostic testing media. The diagnostic test result form includes a POC diagnostic test result generated based on a POC diagnostic test being performed on a patient using a POC diagnostic testing device, a form ID, and a patient identifier, and wherein the POC diagnostic testing media comprises the patient's information, and specimen information.

In 804, application 112 transmits the image to server 102 to extract data from the diagnostic test result form and the POC diagnostic testing media. Server 102 may include POC application 108. POC application 108 may be a web-service.

In 806, application 112 receives the data from server 102. The data includes the patient information, the patient identifier, the form ID, and one or more values corresponding with one or more attributes of the POC diagnostic test result.

In 808, application 112 stores the data in a local storage device. The local storage device may be a non-persistent memory on client device 106, such as cache memory.

In 810, application 112 receives an identification number and date of birth information corresponding to the patient. The identification number may be an accession number. The accession number may correspond with the patient identifier. Furthermore, application 112 may identify or derive the identification number from the data. A user may provide the patient's date of birth information.

In 812, application 112 retrieves an order for the POC diagnostic test conducted on the patient using the identification number and the date of birth information. Application 112 may verify the patient's identity based on the identification number and the patient's date of birth information. For example, the order may include the accession number and the patient's date of birth information. Application 112 may attempt to match the accession number and patient's date of birth information with the identification number identified or derived from the data and the patient's date of birth information received from the user.

In 814, application determines a type of POC diagnostic test based on the form ID. The diagnostic test result form may correspond with a specific type of POC diagnostic test. As such, the form ID may be indicative of the type of POC diagnostic test.

In 816, application 112 determines a relationship between the one or more attributes based on the type of POC diagnostic test. For example, application 112 may determine how the one or more values of the one or more attributes are used to generate a POC diagnostic test result for the type of POC diagnostic test.

In 818, application 112 interprets the POC diagnostic test based on the one or more values and the relationship between the one or more attributes. As a non-limiting example, the POC diagnostic test result may be “negative”, “positive”, or “invalid.”

In 820, application 112 outputs the interpreted POC diagnostic test result. The output may include a visual indicator corresponding to the interpreted POC diagnostic test result. For example, the background of the output may be green for when the POC diagnostic test result is “negative.” Furthermore, the background of the output may be red for when the POC diagnostic test result is “positive.” Moreover, the background of the output may be grey for when the POC diagnostic test result is “invalid.”

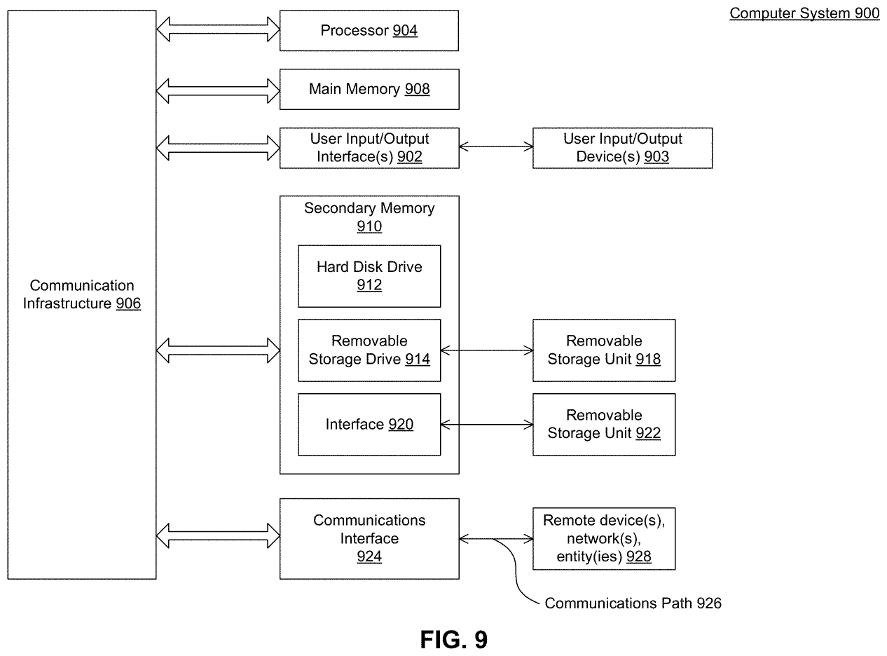

Various embodiments can be implemented, for example, using one or more computer systems, such as computer system 900 shown in FIG. 9. Computer system 900 can be used, for example, to implement methods 200 of FIG. 2A, 250, 600 of FIG. 6, 700 of FIG. 7, and 800 of FIG. 8. Furthermore, computer system 900 can be at least part of server 102, database 104, client device 106, and user device 107, as shown in FIG. 1. For example, computer system 900 routes communication to various applications. Computer system 900 can be any computer capable of performing the functions described herein.

Computer system 900 can be any well-known computer capable of performing the functions described herein.

Computer system 900 includes one or more processors (also called central processing units, or CPUs), such as a processor 904. Processor 904 is connected to a communication infrastructure or bus 906.

One or more processors 904 may each be a graphics processing unit (GPU). In an embodiment, a GPU is a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 900 also includes user input/output device(s) 903, such as monitors, keyboards, pointing devices, etc., that communicate with communication infrastructure 906 through user input/output interface(s) 902.

Computer system 900 also includes a main or primary memory 908, such as random access memory (RAM). Main memory 908 may include one or more levels of cache. Main memory 908 has stored therein control logic (i.e., computer software) and/or data.

Computer system 900 may also include one or more secondary storage devices or memory 910. Secondary memory 910 may include, for example, a hard disk drive 912 and/or a removable storage device or drive 914. Removable storage drive 914 may be a floppy disk drive, a magnetic tape drive, a compact disk drive, an optical storage device, tape backup device, and/or any other storage device/drive.

Removable storage drive 914 may interact with a removable storage unit 918. Removable storage unit 918 includes a computer-usable or readable storage device having stored thereon computer software (control logic) and/or data. Removable storage unit 918 may be a floppy disk, magnetic tape, compact disk, DVD, optical storage disk, and/ any other computer data storage device. Removable storage drive 914 reads from and/or writes to removable storage unit 918 in a well-known manner.

According to an exemplary embodiment, secondary memory 910 may include other means, instrumentalities, or other approaches for allowing computer programs and/or other instructions and/or data to be accessed by computer system 900. Such means, instrumentalities, or other approaches may include, for example, a removable storage unit 922 and an interface 920. Examples of the removable storage unit 922 and the interface 920 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 900 may further include a communication or network interface 924. Communication interface 924 enables computer system 900 to communicate and interact with any combination of remote devices, remote networks, remote entities, etc. (individually and collectively referenced by reference number 928). For example, communication interface 924 may allow computer system 900 to communicate with remote devices 928 over communications path 926, which may be wired and/or wireless, and which may include any combination of LANs, WANs, the Internet, etc. Control logic and/or data may be transmitted to and from computer system 900 via communication path 926.

In an embodiment, a tangible, non-transitory apparatus or article of manufacture comprising a tangible, non-transitory computer useable or readable medium having control logic (software) stored thereon is also referred to herein as a computer program product or program storage device. This includes, but is not limited to, computer system 900, main memory 908, secondary memory 910, and removable storage units 918 and 922, as well as tangible articles of manufacture embodying any combination of the foregoing. Such control logic, when executed by one or more data processing devices (such as computer system 900), causes such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 9. In particular, embodiments can operate with software, hardware, and/or operating system implementations other than those described herein.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

What is claimed is:

1. A computer-implemented method for linking a point of care (POC) testing media and a diagnostic test result form, the method comprising:
receiving an image of the diagnostic test result form and a POC diagnostic testing media, wherein the diagnostic test result form comprises a POC diagnostic test result generated based on a POC diagnostic test being performed on a patient using a POC diagnostic testing device, a form ID, and a patient identifier, and wherein the POC diagnostic testing media comprises the patient's information and specimen information;
confirming that the diagnostic test result form and the POC diagnostic testing media are positioned in a predetermined position such that the patient's information and the patient identifier are visible in the image;
verifying that the diagnostic test result form and the POC diagnostic testing media correspond to the patient;
extracting the form ID on the diagnostic test result form from the image;
extracting one or more values on the diagnostic test result form from the image, wherein the one or more values correspond with one or more attributes tested using the POC diagnostic testing device; and
transmitting the patient information, the patient identifier, the form ID, and the one or more values to a client device.

2. The computer-implemented method of claim 1, wherein verifying that the diagnostic test result form and the POC diagnostic testing media correspond to the patient comprises:
determining that the patient's information and the patient identifier correspond to the patient.

3. The computer-implemented method of claim 1, further comprising:
identifying an existing record of the patient based on the patient's information or the patient identifier; and
correlating the interpreted diagnostic test result with the existing record.

4. The computer-implemented method of claim 1, wherein the POC diagnostic test result is generated based on the one or more values.

5. The method of claim 1, further comprising:
verifying that the POC diagnostic testing media and the diagnostic test result form corresponds to the same patient by:
extracting the patient identifier from the diagnostic test result form; and
determining that the patient's information and the patient identifier correspond to the same patient.

6. The computer-implemented method of claim 1, wherein extracting the one or more values on the diagnostic test result form from the image using optical character recognition.

7. The computer-implemented method of claim 1, wherein extracting the one or more values using a machine learning algorithm trained to identify and extract the one or more values from the diagnostic test result form.

8. The computer-implemented method of claim 1, wherein the POC diagnostic testing media comprises an assay for a nucleocapsid protein antigen of SARS-CoV-2, Immunoglobulin M (IgM)/Immunoglobulin G (IgG) antibodies to SARS-CoV-2 spike protein, SARS-CoV-2 nucleotides coding for SARS-CoV2-1 spike protein or SARS-CoV2-2 membrane protein, or a combination thereof.

9. The computer-implemented method of claim 1, wherein the POC diagnostic testing media comprises an assay for one or more of a hemagglutinin antigen (HA), neuraminidase antigen (NA), nucleocapsid antigen (NA), matrix (M), or nucleocapsid proteins (NP).

10. A system for linking a point of care (POC) testing media and a diagnostic test result form, the system comprising:
a memory;
at least one processor coupled to the memory, wherein the at least processor is configured to:
receive an image of a diagnostic test result form and a POC diagnostic testing media, wherein the diagnostic test result form comprises a POC diagnostic test result generated based on a POC diagnostic test being performed on a patient using a POC diagnostic testing device, a form ID, and a patient identifier, and wherein the POC diagnostic testing media comprises the patient's information, and specimen information;
confirm that the diagnostic test result form and the POC diagnostic testing media are positioned in a predetermined position such that the patient's information and the patient identifier are visible in the image;
verify that the diagnostic test result form and the POC diagnostic testing media correspond to the patient;
extract the form ID on the diagnostic test result form from the image;
extract one or more values on the diagnostic test result form from the image, wherein the one or more values correspond with one or more attributes tested using the POC diagnostic testing device; and
transmit the patient information, the patient identifier, the form ID, and the one or more values to a client device.

11. The system of claim 10, wherein verifying that the diagnostic test result form and the POC diagnostic testing media correspond to the patient comprises:
determining that the patient's information and the patient identifier correspond to the patient.

12. The system of claim 10, wherein the at least one processor is configured to:
identify an existing record of the patient based on the patient's information or the patient identifier; and
correlate the interpreted diagnostic test result with the existing record.

13. The system of claim 10, wherein the POC diagnostic test result is generated using the one or more values.

14. The system of claim 10, wherein the at least one processor is configured to:
verify that the POC diagnostic testing media and the diagnostic test result form corresponds to the same patient by:
extract the patient identifier from the diagnostic test result form; and
determine that the patient's information and the patient identifier correspond to the same patient.

15. The system of claim 10, wherein extracting the one or more values on the diagnostic test result form from the image using optical character recognition.

16. The system of claim 10, wherein extracting the one or more values using a machine learning algorithm trained to identify and extract the one or more values from the diagnostic test result form.

17. The system of claim 10, wherein the POC diagnostic testing media comprises an assay for a nucleocapsid protein antigen of SARS-CoV-2, Immunoglobulin M (IgM)/Immunoglobulin G (IgG) antibodies to SARS-CoV-2 spike protein, SARS-CoV-2 nucleotides coding for SARS-CoV2-1 spike protein or SARS-CoV2-2 membrane protein, or a combination thereof.

18. The system of claim 10, wherein the POC diagnostic testing media comprises an assay for one or more of a hemagglutinin antigen (HA), neuraminidase antigen (NA), nucleocapsid antigen (NA), matrix (M), or nucleocapsid proteins (NP).

19. A non-transitory computer-readable medium having instructions stored thereon, execution of which, by one or more processors of a device, cause the one or more processors to perform operations comprising:
receiving an image of a diagnostic test result form and a POC diagnostic testing media, wherein the diagnostic test result form comprises a POC diagnostic test result generated based on a POC diagnostic test being performed on a patient using a POC diagnostic testing device, a form ID, and a patient identifier, and wherein the POC diagnostic testing media comprises the patient's information, and specimen information;
confirming that the diagnostic test result form and the POC diagnostic testing media are positioned in a predetermined position such that the patient's information and the patient identifier are visible in the image;
verifying that the diagnostic test result form and the POC diagnostic testing media correspond to the patient;
extracting the form ID on the diagnostic test result form from the image;
extracting one or more values on the diagnostic test result form from the image, wherein the one or more values correspond with one or more attributes tested using the POC diagnostic testing device; and
transmitting the patient information, the patient identifier, the form ID, and the one or more values to a client device.

20. The non-transitory computer-readable medium of claim 19, wherein verifying that the diagnostic test result form and the POC diagnostic testing media correspond to the patient comprises:
determining that the patient's information and the patient identifier correspond to the patient.

21. The non-transitory computer-readable medium of claim 19, wherein the operations further comprising:
identifying an existing record of the patient based on the patient's information or the patient identifier; and
correlating the interpreted diagnostic test result with the existing record.

22. The non-transitory computer-readable medium of claim 19, wherein the POC diagnostic test result is generated using the one or more values.

23. The non-transitory computer-readable medium of claim 19, wherein extracting the one or more values on the diagnostic test result form from the image using optical character recognition.

24. The non-transitory computer-readable medium of claim 19, wherein extracting the one or more values using a machine learning algorithm trained to identify and extract the one or more values from the diagnostic test result form.

25. The non-transitory computer-readable medium of claim 19, wherein the POC diagnostic testing media comprises an assay for a nucleocapsid protein antigen of SARS-CoV-2, Immunoglobulin M (IgM)/Immunoglobulin G (IgG) antibodies to SARS-CoV-2 spike protein, SARS-CoV-2 nucleotides coding for SARS-CoV2-1 spike protein or SARS-CoV2-2 membrane protein, or a combination thereof.

26. The non-transitory computer-readable medium of claim 19, wherein the POC diagnostic testing media comprises an assay for one or more of a hemagglutinin antigen (HA), neuraminidase antigen (NA), nucleocapsid antigen (NA), matrix (M), or nucleocapsid proteins (NP).

27. A device configured to interpret a Point Of Care (POC) diagnostic test result, the device comprising:
at least one memory;
at least one camera;
at least one processor coupled to the at least one memory and the at least one camera, the at least one processor configured to:
cause the at least one camera to capture an image of a diagnostic test result form and a POC diagnostic testing media, wherein the diagnostic test result form comprises a POC diagnostic test result generated based on a POC diagnostic test being performed on a patient using a POC diagnostic testing device, a form ID, and a patient identifier, and wherein the POC diagnostic testing media comprises the patient's information, and specimen information;
transmit the image to a web-service to extract data from the diagnostic test result form and the POC diagnostic testing media;
receive the data from the web-service, wherein the data includes the patient information, the patient identifier, the form ID, and one or more values corresponding with one or more attributes of the POC diagnostic test result;
store the data in the memory;
receive an identification number and date of birth information corresponding to the patient;
retrieve an order for the POC diagnostic test conducted on the patient using the identification number and the date of birth information;
determine a type of POC diagnostic test based on the form ID;
determine a relationship between the one or more attributes based on the type of POC diagnostic test;
interpret the POC diagnostic test result based on the one or more values and the relationship between the one or more attributes; and
output the interpreted POC diagnostic test result.

28. The device of claim 27, wherein the POC diagnostic testing media comprises an assay for a nucleocapsid protein antigen of SARS-CoV-2, Immunoglobulin M (IgM)/Immunoglobulin G (IgG antibodies to SARS-CoV-2 spike protein, SARS-CoV-2 nucleotides coding for SARS-CoV2-1 spike protein or SARS-CoV2-2 membrane protein, or a combination thereof.

29. The device of claim 27, wherein the POC diagnostic testing media comprises an assay for one or more of a hemagglutinin antigen (HA), neuraminidase antigen (NA), nucleocapsid antigen (NA), matrix (M), or nucleocapsid proteins (NP).
